(19) United States
(12) Patent Application Publication
(10) Pub. No.: US 2020/0276204 A1
(43) Pub. Date: Sep. 3, 2020

(54) COMBINATION THERAPY FOR IMMUNOLOGICAL DISEASES

(71) Applicant: KEZAR LIFE SCIENCES, South San Francisco, CA (US)

(72) Inventors: Dustin McMinn, Pacifica, CA (US); Tony Muchamuel, Boulder Creek, CA (US); Henry Johnson, San Bruno, CA (US); Eric Lowe, Moraga Town, CA (US); Janet Anderl, Pacifica, CA (US)

(21) Appl. No.: 16/645,567

(22) PCT Filed: Sep. 21, 2018
(86) PCT No.: PCT/US18/52105

§ 371 Date: Mar. 9, 2020

Related U.S. Application Data
(60) Provisional application No. 62/561,369, filed on Sep. 21, 2017.

(51) Int. Cl. A61K 31/5377; A61K 31/397; A61K 31/4025; A61K 31/4427; A61K 31/4523; A61K 31/336; A61K 31/4178; A61K 31/351; A61K 47/40; A61K 47/26; A61P 19/02

(52) U.S. Cl. CPC A61K 31/5377; A61K 31/397; A61K 31/4025; A61K 31/4427; A61K 31/4523; A61K 9/0019; A61K 31/4178; A61K 31/351; A61K 47/40; A61K 47/26; A61P 19/02; A61K 31/336

(57) ABSTRACT

Provided herein are methods of treating immune-related disorders comprising administering to a patient suffering from the disorder a therapeutically effective amount of a LMP2-selective inhibitor and a therapeutically effective amount of a LMP7-selective inhibitor. Also provided herein are compositions comprising a LMP2-selective inhibitor and a LMP7-selective inhibitor.

### BACKGROUND

### Field of the Invention

The disclosure relates to methods of treating immune-related disorders in a patient comprising administering to the patient, in combination, a therapeutically effective amount of a LMP2-selective inhibitor and a therapeutically effective amount of a LMP7-selective inhibitor. The disclosure further relates to pharmaceutical compositions comprising an LMP2-selective inhibitor and an LMP7-selective inhibitor.

### Description of Related Technology

In eukaryotes, protein degradation is predominately mediated through the ubiquitin proteasome pathway, in which proteins targeted for destruction are ligated to the 76 amino acid polypeptide ubiquitin. Once targeted, ubiquitinated proteins serve as substrates for the 26S proteasome, a multicatalytic protease, which cleaves proteins into short peptides through the action of its three major proteolytic activities. While having a general function in intracellular protein turnover, proteasome-mediated degradation also plays a key role in many processes such as major histocompatibility complex (MHC) class I antigen presentation, apoptosis, cell growth regulation, NF-κB activation, antigen processing, and transduction of pro-inflammatory signals.

The 20S proteasome is a 700 kDa cylindrical-shaped multicatalytic protease complex composed of 28 subunits organized into four rings. In yeast and other eukaryotes, seven different a subunits form the outer rings, and seven different β subunits comprise the inner rings. The α subunits serve as binding sites for the 19S (PA700) and 11S (PA28) regulatory complexes, as well as a physical barrier for the inner proteolytic chamber formed by the two β subunit rings. Thus, in vivo, the proteasome is believed to exist as a 26S particle (“the 26S proteasome”). In vivo experiments have shown that inhibition of the 20S form of the proteasome can be readily correlated to inhibition of 26S proteasome. Cleavage of amino-terminal prosequences of active site β subunits during particle formation expose amino-terminal threonine residues, which serve as the catalytic nucleophiles. The subunits responsible for catalytic activity in proteasomes thus possess an amino terminal nucleophilic residue, and these subunits belong to the family of N-terminal nucleophile (Ntn) hydrolases (where the nucleophilic N-terminal residue is, for example, Cys, Ser, Thr, and other nucleophilic moieties). This family includes, for example, penicillin G acylase (PGA), penicillin V acylase (PVA), glutamine PRPP amidotransferase (GAT), and bacterial glycosylasparaginase. Through the use of different peptide substrates, three major proteolytic activities have been defined for the eukaryote 20S proteasome: chymotrypsin-like activity (CT-L), which cleaves after large hydrophobic residues; trypsin-like activity (T-L), which cleaves after basic residues; and peptidylglutamyl peptide hydrolyzing activity (PGPH) or caspase-like (C-L), which cleaves after acidic residues. In mammals, most cells and tissues express a “constitutive proteasome” in which the three active sites are β5, β1, and β2, which encode CT-L, C-L, and T-L activities, respectively. Higher order vertebrates also possess three interferon-γ-inducible β subunits (LMP7, LMP2 and MECL1), which replace their constitutive proteasome counterparts, β5, β1 and β2 respectively, thus altering the catalytic activities of the proteasome. The major proteasome proteolytic activities appear to be contributed by different catalytic sites because inhibitors, point mutations in β subunits, and the exchange of γ interferon-inducing β subunits alter these activities to various degrees.

### SUMMARY

In one aspect, the disclosure provides a pharmaceutical composition comprising: (a) an LMP2-selective inhibitor, (b) an LMP7-selective inhibitor; and (c) a pharmaceutically acceptable carrier.

In another aspect, the disclosure provides a method of treating an immune-related disorder in a patient comprising administering to the patient in combination: (a) a therapeutically effective amount of an LMP2-selective inhibitor, and (b) a therapeutically effective amount of an LMP7-selective inhibitor. In some cases, the immune-related disorder is selected from the group consisting of rheumatoid arthritis, lupus, multiple sclerosis, psoriasis, chronic obstructive pulmonary disease (“COPD”), granulomatosis and vasculitis, graft or transplant-related disease, and fibrotic disease. In some embodiments, the disorder is rheumatoid arthritis, lupus, multiple sclerosis, psoriasis, COPD, or granulomatosis and vasculitis. In various cases, the lupus is systemic lupus erythematosus (“SLE”), subacute cutaneous lupus, drug-induced lupus, neonatal lupus, or discoid lupus. In some cases, the fibrotic disease is selected from the group consisting of cystic fibrosis, idiopathic pulmonary fibrosis (“IPF”), cirrhosis, biliary atresia, atrial fibrosis, endomyocardial fibrosis, arterial stiffness, arthrofibrosis, Crohn's Disease, Dupuytren's contracture, keloid, mediastinal fibrosis, myelofibrosis, Peyronie's disease, nephrogenic systemic fibrosis, progressive massive fibrosis, retroperitoneal fibrosis, and scleroderma. In some embodiments, the LMP2-selective inhibitor and the LMP7-selective inhibitor are administered in the same pharmaceutical composition. In various embodiments, the LMP2-selective inhibitor and the LMP7-selective inhibitor are administered in separate pharmaceutical compositions.

The LMP2-selective inhibitor disclosed herein can have a structure of Formula (II), or a pharmaceutically acceptable salt thereof:

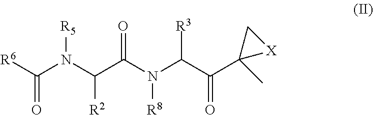

wherein: X is selected from O, S, NH, and N—C₁₋₆alkyl; R² and R³ are each independently selected from aryl, C₁₋₆aralkyl, heteroaryl, and C₁₋₆heteroaralkyl; R⁵ is selected from hydrogen, OH, C₁₋₆aralkyl, and C₁₋₆alkyl; R⁶ is heteroaryl, piperidinyl, piperazinyl, morpholinyl, a lactone, a lactam, or

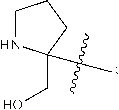 [SMILES: *C1(CO)CCCN1]

and R⁸ is selected from hydrogen, C₁₋₆alkyl, and C₁₋₆aralkyl.

In some embodiments, the LMP2-selective inhibitor has a structure selected from the group consisting of:

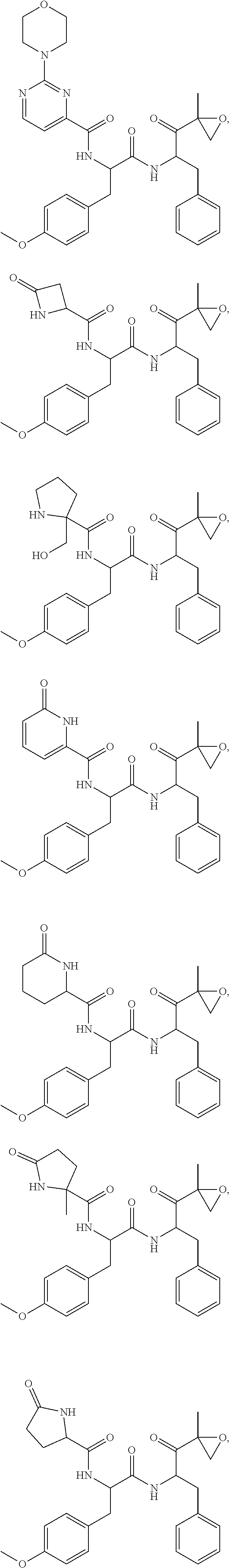

and a combination thereof, or a pharmaceutically acceptable salt thereof. In some cases, the LMP2-selective inhibitor is selected from the group consisting of:

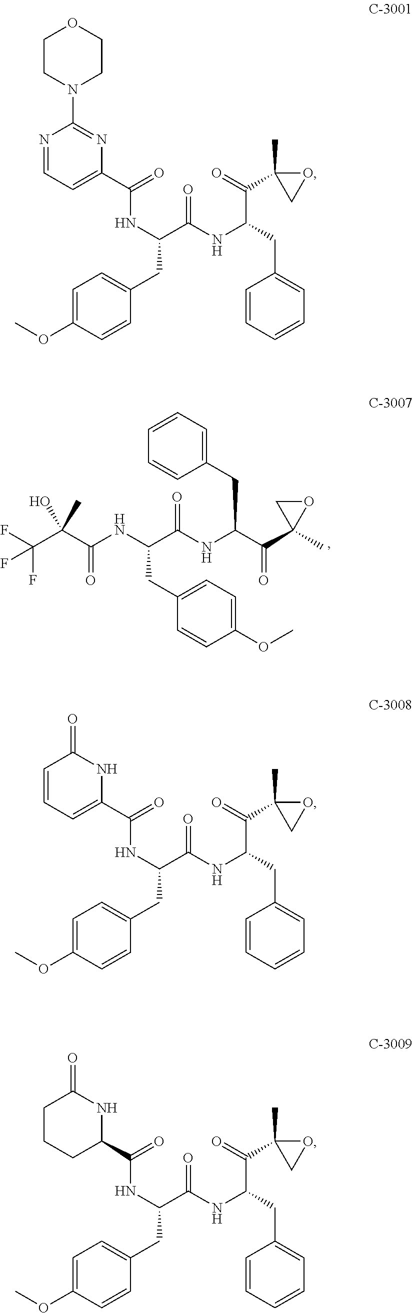

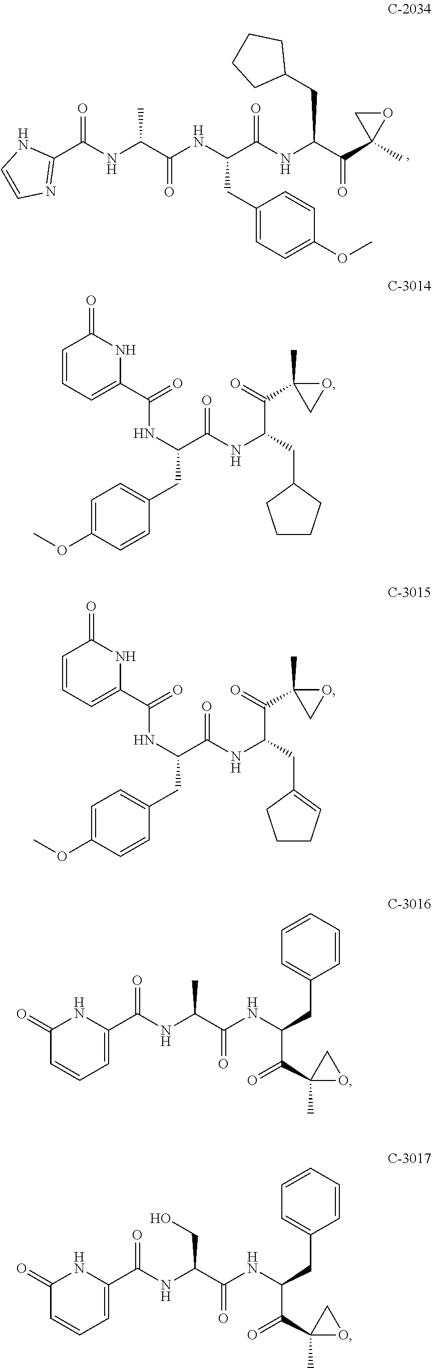

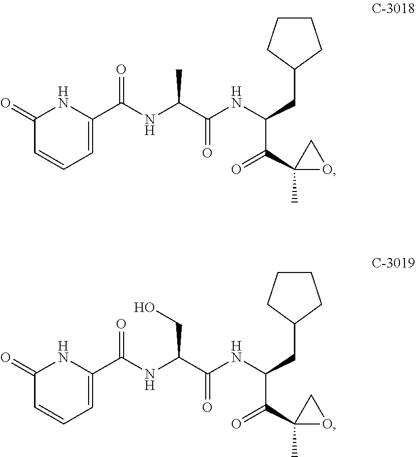 [SMILES: C[C@H](NC(=O)c1cccc(=O)[nH]1)C(=O)N[C@@H](CC1CCCC1)C(=O)[C@@]1(C)CO1 ; C[C@]1(C(=O)[C@H](CC2CCCC2)NC(=O)[C@H](CO)NC(=O)c2cccc(=O)[nH]2)CO1]

and a combination thereof, or a pharmaceutically acceptable salt thereof.

In some cases, the LMP2-selective inhibitor is

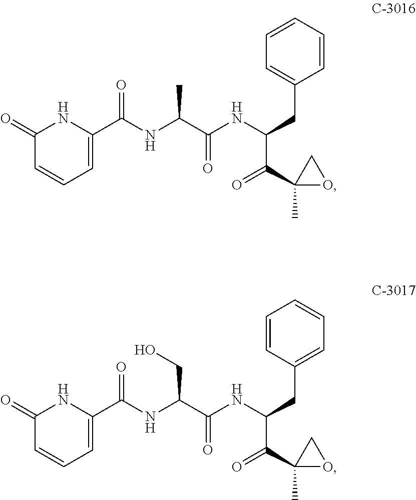 [SMILES: C[C@H](NC(=O)c1cccc(=O)[nH]1)C(=O)N[C@@H](Cc1ccccc1)C(=O)[C@@]1(C)CO1 ; C[C@]1(C(=O)[C@H](Cc2ccccc2)NC(=O)[C@H](CO)NC(=O)c2cccc(=O)[nH]2)CO1]

or a combination thereof, or a pharmaceutically acceptable salt thereof.

The LMP7-selective inhibitor can have a structure of Formula (X), or a pharmaceutically acceptable salt thereof:

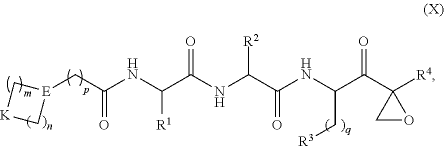

wherein: m and n each independently are 0, 1 or 2, and m+n=2, 3, or 4; p is 0 or 1; q is 0, 1, or 2; K is selected from the group consisting of CR⁵R⁶, NR⁷, N(C═O)0R⁷, —NH— (C═O)—, O, S, SO, and SO₂; E is N or CR⁷; R¹ is selected from the group consisting of H, C₁₋₆alkyl, C₂₋₆alkenyl, C₂₋₆alkynyl, C₃₋₆cycloalkyl, and 3-6 membered heterocycloalkyl, wherein R¹ is optionally substituted with one or more substituents selected from the group consisting of halo, OR⁷, SR⁷, N(R⁷)₂, CN, and (C═O)N(R⁷)₂; R² is C₁₋₂alkylene-G or (C═O)-G; wherein G is selected from the group consisting of aryl, heteroaryl, and pyridinone, with the proviso that when R² is CH₂phenyl, the phenyl is substituted with one or more substituents selected from the group consisting of OR⁷, halo, C₁₋₃alkyl, OCF₃, SO₂R⁷, (C═O)N(R⁷)₂, CN, and SO₂N(R⁷)₂; R³ is non-aromatic and selected from the group consisting of C₃₋₇cycloalkyl, C₃₋₇cycloalkenyl, a 3-7 membered heterocycloalkyl, and a 3-7 membered heterocycloalkenyl, wherein R³ is optionally substituted with one or more substituents selected from the group consisting of halo, ═O, OR⁷, SR⁷, N(R⁷)₂, O(C═O)N(R⁷)₂, and C₁₋₆alkyl; R⁴ is H or C₁₋₃alkyl; R⁵ and R⁶ are each independently selected from the group consisting of H, OH, halo, C₁₋₃alkyl, and CF₃, or R⁵ and R⁶ together with the carbon to which they are attached form C═O or

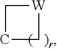

wherein W is O or NR⁷, and r is 1, 2 or 3; and each R⁷ is independently H or C₁₋₆alkyl.

In some cases, the LMP7-selective inhibitor has a structure selected from group consisting of:

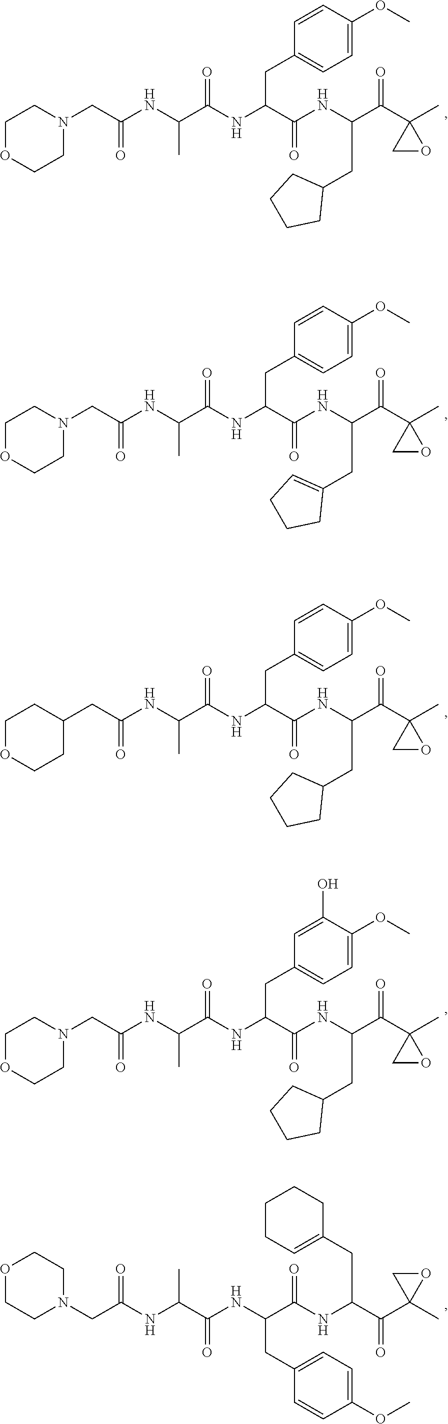 [SMILES: COc1ccc(CC(NC(=O)C(C)NC(=O)CN2CCOCC2)C(=O)NC(CC2CCCC2)C(=O)C2(C)CO2)cc1 ; COc1ccc(CC(NC(=O)C(C)NC(=O)CN2CCOCC2)C(=O)NC(CC2=CCCC2)C(=O)C2(C)CO2)cc1 ; COc1ccc(CC(NC(=O)C(C)NC(=O)CC2CCOCC2)C(=O)NC(CC2CCCC2)C(=O)C2(C)CO2)cc1 ; COc1ccc(CC(NC(=O)C(C)NC(=O)CN2CCOCC2)C(=O)NC(CC2CCCC2)C(=O)C2(C)CO2)cc1O ; COc1ccc(CC(NC(=O)C(C)NC(=O)CN2CCOCC2)C(=O)NC(CC2=CCCCC2)C(=O)C2(C)CO2)cc1]

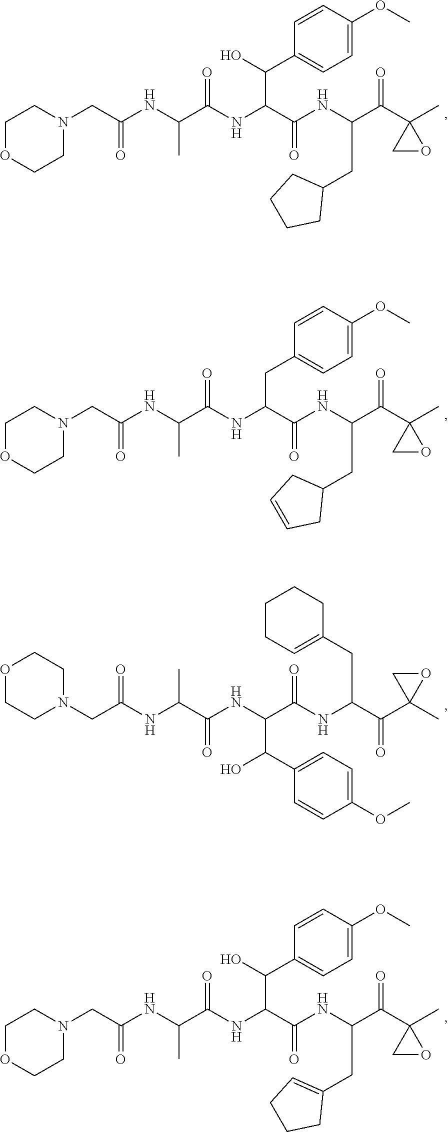 [SMILES: COc1ccc(C(O)C(NC(=O)C(C)NC(=O)CN2CCOCC2)C(=O)NC(CC2CCCC2)C(=O)C2(C)CO2)cc1 ; COc1ccc(CC(NC(=O)C(C)NC(=O)CN2CCOCC2)C(=O)NC(CC2CC=CC2)C(=O)C2(C)CO2)cc1 ; COc1ccc(C(O)C(NC(=O)C(C)NC(=O)CN2CCOCC2)C(=O)NC(CC2=CCCCC2)C(=O)C2(C)CO2)cc1 ; COc1ccc(C(O)C(NC(=O)C(C)NC(=O)CN2CCOCC2)C(=O)NC(CC2=CCCC2)C(=O)C2(C)CO2)cc1]

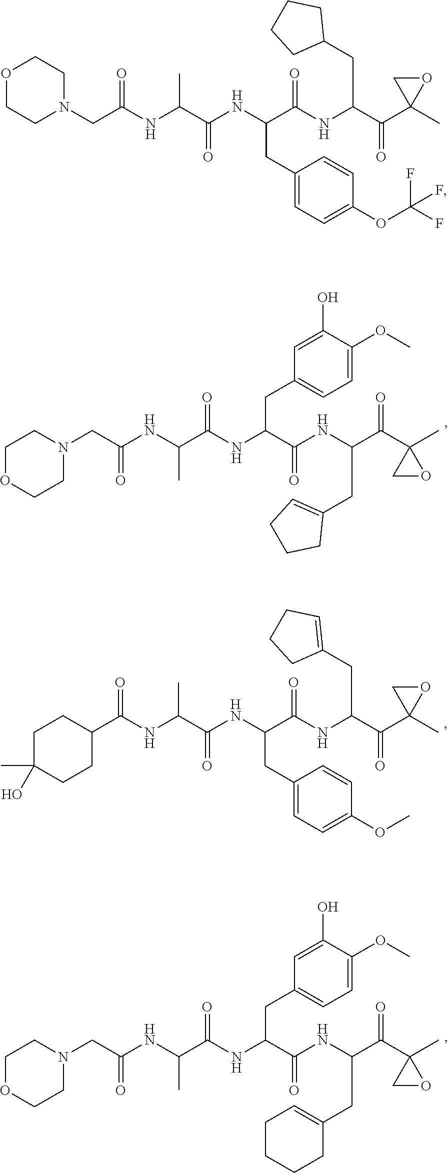

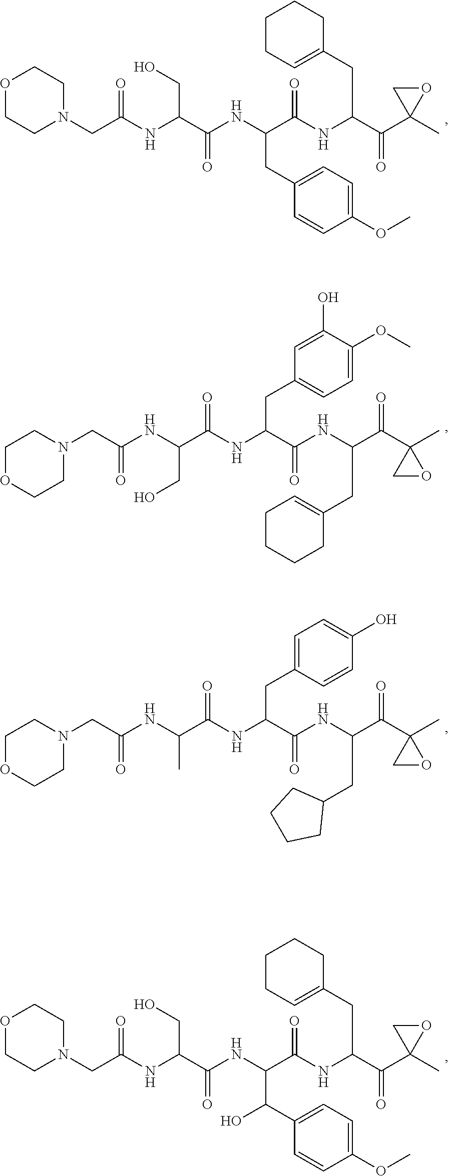 [SMILES: COc1ccc(CC(NC(=O)C(CO)NC(=O)CN2CCOCC2)C(=O)NC(CC2=CCCCC2)C(=O)C2(C)CO2)cc1 ; COc1ccc(CC(NC(=O)C(CO)NC(=O)CN2CCOCC2)C(=O)NC(CC2=CCCCC2)C(=O)C2(C)CO2)cc1O ; CC(NC(=O)CN1CCOCC1)C(=O)NC(Cc1ccc(O)cc1)C(=O)NC(CC1CCCC1)C(=O)C1(C)CO1 ; COc1ccc(C(O)C(NC(=O)C(CO)NC(=O)CN2CCOCC2)C(=O)NC(CC2=CCCCC2)C(=O)C2(C)CO2)cc1]

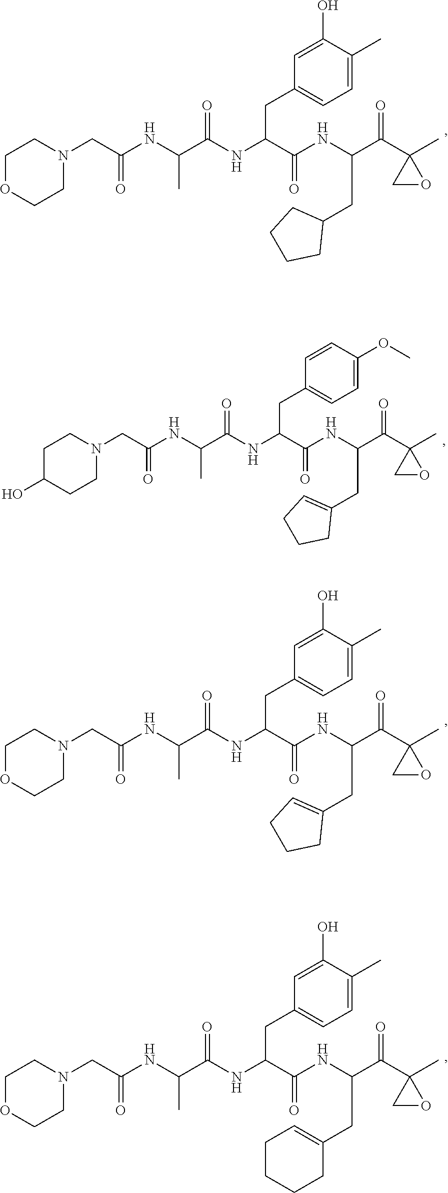 [SMILES: Cc1ccc(CC(NC(=O)C(C)NC(=O)CN2CCOCC2)C(=O)NC(CC2CCCC2)C(=O)C2(C)CO2)cc1O ; COc1ccc(CC(NC(=O)C(C)NC(=O)CN2CCC(O)CC2)C(=O)NC(CC2=CCCC2)C(=O)C2(C)CO2)cc1 ; Cc1ccc(CC(NC(=O)C(C)NC(=O)CN2CCOCC2)C(=O)NC(CC2=CCCC2)C(=O)C2(C)CO2)cc1O ; Cc1ccc(CC(NC(=O)C(C)NC(=O)CN2CCOCC2)C(=O)NC(CC2=CCCCC2)C(=O)C2(C)CO2)cc1O]

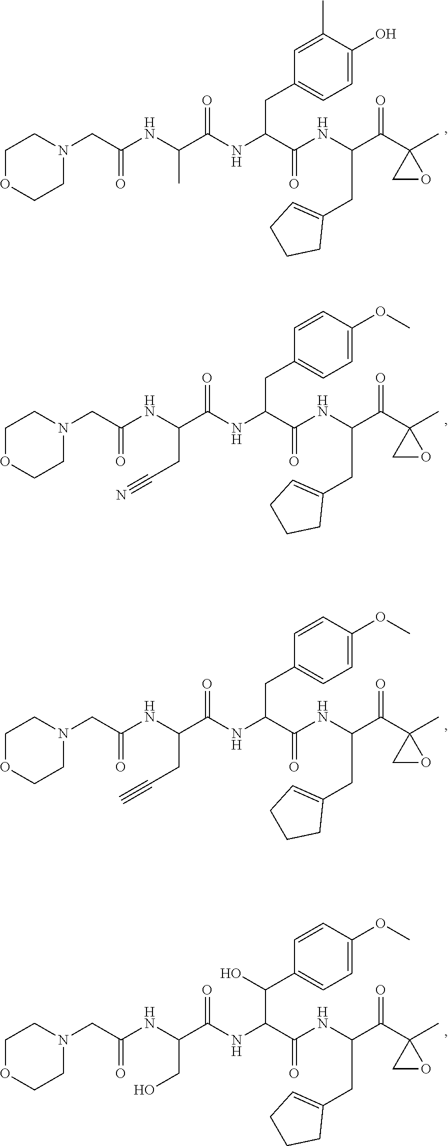 [SMILES: Cc1cc(CC(NC(=O)C(C)NC(=O)CN2CCOCC2)C(=O)NC(CC2=CCCC2)C(=O)C2(C)CO2)ccc1O ; COc1ccc(CC(NC(=O)C(CC#N)NC(=O)CN2CCOCC2)C(=O)NC(CC2=CCCC2)C(=O)C2(C)CO2)cc1 ; C#CCC(NC(=O)CN1CCOCC1)C(=O)NC(Cc1ccc(OC)cc1)C(=O)NC(CC1=CCCC1)C(=O)C1(C)CO1 ; COc1ccc(C(O)C(NC(=O)C(CO)NC(=O)CN2CCOCC2)C(=O)NC(CC2=CCCC2)C(=O)C2(C)CO2)cc1]

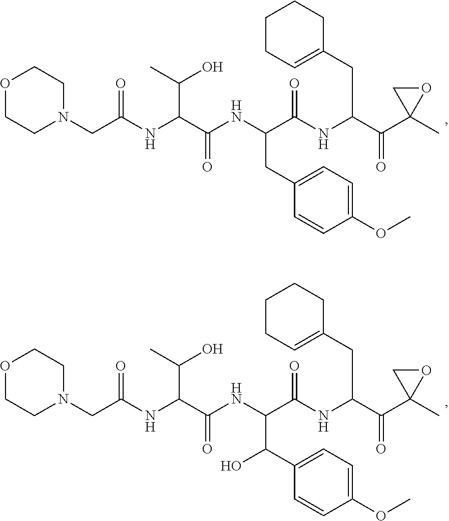 [SMILES: COc1ccc(CC(NC(=O)C(NC(=O)CN2CCOCC2)C(C)O)C(=O)NC(CC2=CCCCC2)C(=O)C2(C)CO2)cc1 ; COc1ccc(C(O)C(NC(=O)C(NC(=O)CN2CCOCC2)C(C)O)C(=O)NC(CC2=CCCCC2)C(=O)C2(C)CO2)cc1]

and a combination thereof, or a pharmaceutically acceptable salt thereof.

In various embodiments, the LMP7-selective inhibitor is selected from the group consisting of:

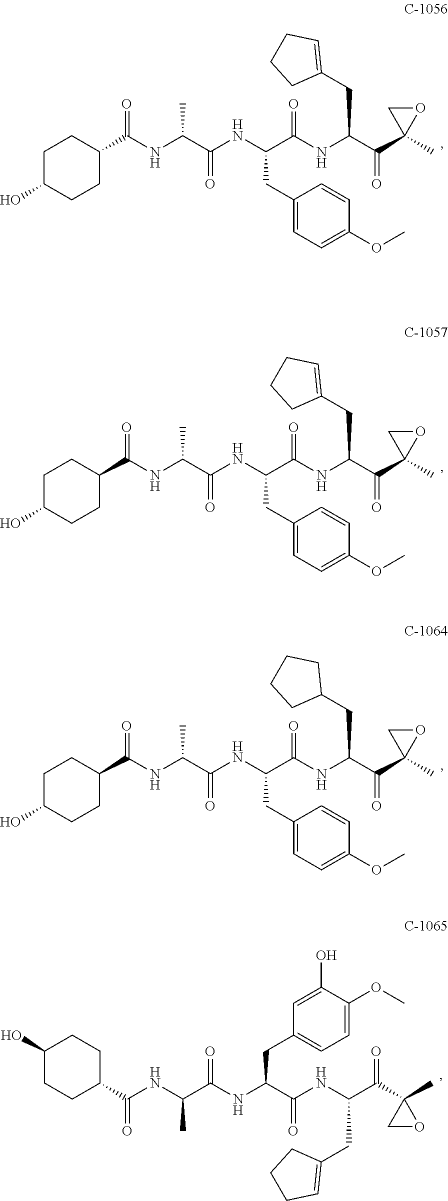 [SMILES: COc1ccc(C[C@H](NC(=O)[C@@H](C)NC(=O)[C@H]2CC[C@@H](O)CC2)C(=O)N[C@@H](CC2=CCCC2)C(=O)[C@@]2(C)CO2)cc1 ; COc1ccc(C[C@H](NC(=O)[C@@H](C)NC(=O)[C@H]2CC[C@H](O)CC2)C(=O)N[C@@H](CC2=CCCC2)C(=O)[C@@]2(C)CO2)cc1 ; COc1ccc(C[C@H](NC(=O)[C@@H](C)NC(=O)[C@H]2CC[C@H](O)CC2)C(=O)N[C@@H](CC2CCCC2)C(=O)[C@@]2(C)CO2)cc1 ; COc1ccc(C[C@H](NC(=O)[C@@H](C)NC(=O)[C@H]2CC[C@H](O)CC2)C(=O)N[C@@H](CC2=CCCC2)C(=O)[C@@]2(C)CO2)cc1O]

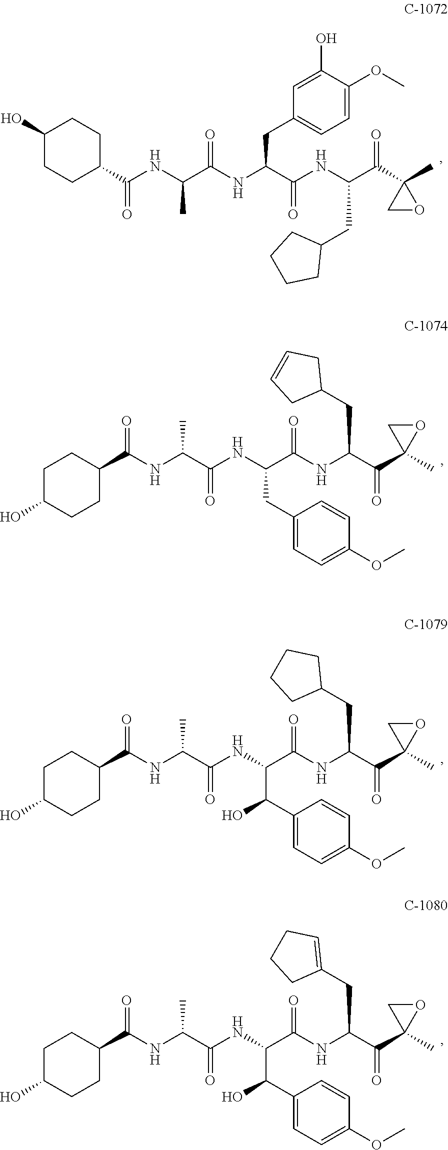 [SMILES: COc1ccc(C[C@H](NC(=O)[C@@H](C)NC(=O)[C@H]2CC[C@H](O)CC2)C(=O)N[C@@H](CC2CCCC2)C(=O)[C@@]2(C)CO2)cc1O ; COc1ccc(C[C@H](NC(=O)[C@@H](C)NC(=O)[C@H]2CC[C@H](O)CC2)C(=O)N[C@@H](CC2CC=CC2)C(=O)[C@@]2(C)CO2)cc1 ; COc1ccc([C@@H](O)[C@H](NC(=O)[C@@H](C)NC(=O)[C@H]2CC[C@H](O)CC2)C(=O)N[C@@H](CC2CCCC2)C(=O)[C@@]2(C)CO2)cc1 ; COc1ccc([C@@H](O)[C@H](NC(=O)[C@@H](C)NC(=O)[C@H]2CC[C@H](O)CC2)C(=O)N[C@@H](CC2=CCCC2)C(=O)[C@@]2(C)CO2)cc1]

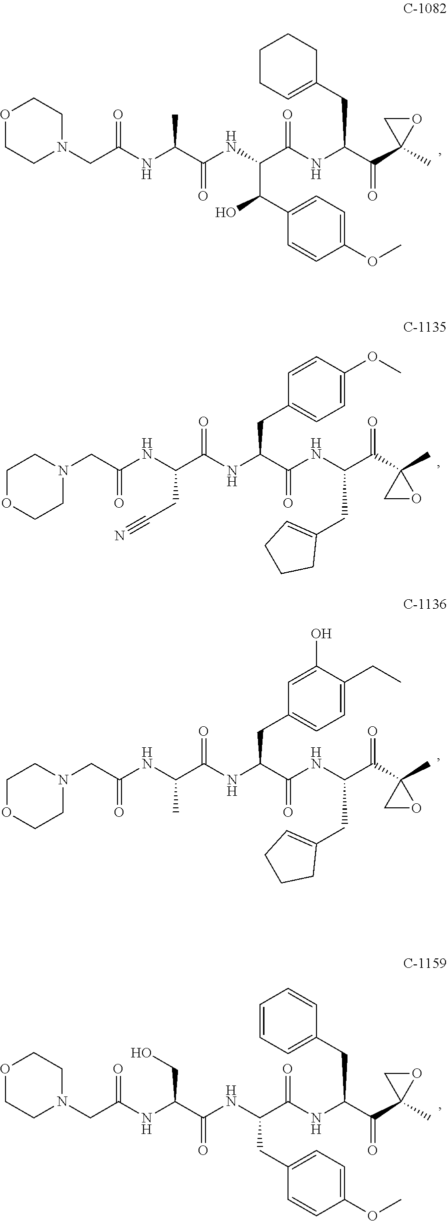 [SMILES: COc1ccc([C@@H](O)[C@H](NC(=O)[C@H](C)NC(=O)CN2CCOCC2)C(=O)N[C@@H](CC2=CCCCC2)C(=O)[C@@]2(C)CO2)cc1 ; COc1ccc(C[C@H](NC(=O)[C@H](CC#N)NC(=O)CN2CCOCC2)C(=O)N[C@@H](CC2=CCCC2)C(=O)[C@@]2(C)CO2)cc1 ; CCc1ccc(C[C@H](NC(=O)[C@H](C)NC(=O)CN2CCOCC2)C(=O)N[C@@H](CC2=CCCC2)C(=O)[C@@]2(C)CO2)cc1O ; COc1ccc(C[C@H](NC(=O)[C@H](CO)NC(=O)CN2CCOCC2)C(=O)N[C@@H](Cc2ccccc2)C(=O)[C@@]2(C)CO2)cc1]

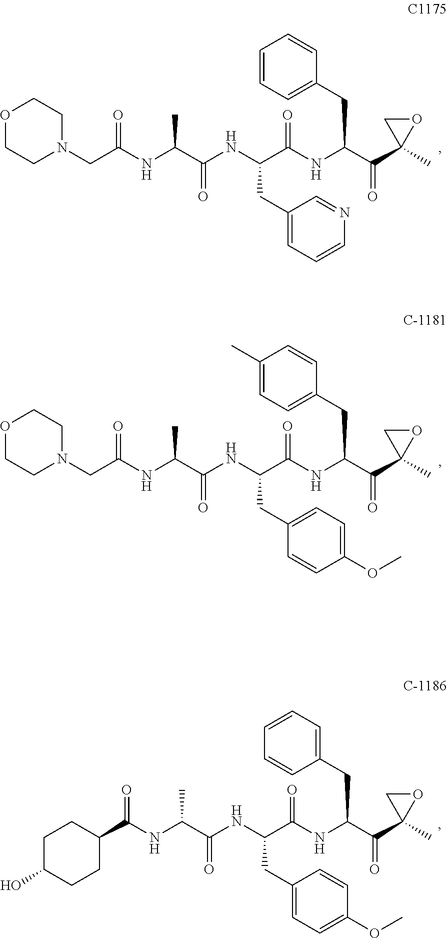 [SMILES: C[C@H](NC(=O)CN1CCOCC1)C(=O)N[C@@H](Cc1cccnc1)C(=O)N[C@@H](Cc1ccccc1)C(=O)[C@@]1(C)CO1 ; COc1ccc(C[C@H](NC(=O)[C@H](C)NC(=O)CN2CCOCC2)C(=O)N[C@@H](Cc2ccc(C)cc2)C(=O)[C@@]2(C)CO2)cc1 ; COc1ccc(C[C@H](NC(=O)[C@@H](C)NC(=O)[C@H]2CC[C@H](O)CC2)C(=O)N[C@@H](Cc2ccccc2)C(=O)[C@@]2(C)CO2)cc1]

and a combination thereof, or a pharmaceutically acceptable salt thereof. In some cases, the LMP7-selective inhibitor is selected from the group consisting of C-1056, C-1057, C-1064, C-1065, C-1072, C-1074, C-1079, C-1080, C-1186, and a combination thereof, or a pharmaceutically acceptable salt thereof.

In some cases, the pharmaceutical composition disclosed herein comprises comprise a cyclodextrin. Suitable cyclodextrins include, for example, beta-cyclodextrin, sulfobutylether-beta-cyclodextrin, or (2-hydroxypropyl)-beta-cyclodextrin.

In various embodiments, the pharmaceutical composition disclosed herein comprises a surfactant. In some embodiments, the surfactant comprises a polysorbate. In some cases, the surfactant comprises polysorbate 80. In some embodiments, the surfactant is present in an amount of about 10% (w/w).

In some cases, the LMP2-selective inhibitor and the LMP7-selective inhibitor are administered simultaneously. In various cases, the LMP2-selective inhibitor and the LMP7-selective inhibitor are administered in the same pharmaceutical composition. In some embodiments, the LMP2-selective inhibitor and the LMP7-selective inhibitor are administered sequentially.

In some cases, the LMP2-selective inhibitor and the LMP7-selective inhibitor are administered orally or parenterally. In some embodiments, at least one of the LMP2-selective inhibitor and the LMP7-selective inhibitor is administered subcutaneously. In various embodiments, each of the LMP2-selective inhibitor and the LMP7-selective inhibitor is administered subcutaneously.

In some embodiments, the LMP2-selective inhibitor and the LMP7-selective inhibitor are administered via injection and at the same injection site. In various embodiments, the LMP2-selective inhibitor and the LMP7-selective inhibitor are administered via injection at different injection sites.

Further aspects and advantages will be apparent to those of ordinary skill in the art from a review of the following detailed description, taken in conjunction with the drawings. The description hereafter includes specific embodiments with the understanding that the disclosure is illustrative, and is not intended to limit the invention to the specific embodiments described herein.

### BRIEF DESCRIPTION OF THE DRAWINGS

FIG. 1A shows the effect on whole blood, kidney, and splenocyte samples of 20 mg/kg and 30 mg/kg of C-1057.

FIG. 1B shows the effect on whole blood, kidney, and splenocyte samples of 5 mg/kg and 10 mg/kg of C-3017.

FIG. 1C shows the effect on whole blood, kidney, and splenocyte samples of the combination of C-3017 and C-1057 at dosages of 20 and 5 mg/kg, respectively and 20 and 10 mg/kg respectively.

FIG. 2 shows the clinical score over time in an arthritis mouse model, where mice were treated with vehicle (open square), C-1057 at 20 mg/kg (filled triangle), C-3017 at 5 mg/kg (filled square), ONX 0914 at 10 mg/kg (open circle), and combination of C-1057 at 20 mg/kg and C-3017 at 5 mg/kg (filled circle).

### DETAILED DESCRIPTION

Provided herein are methods for treating a patient suffering from an immune-related disorder comprising administering to the patient a combination therapy comprising a therapeutically effective amount of a LMP2-selective inhibitor and a therapeutically effective amount of a LMP7-selective inhibitor to treat the immune-related disorder. As used herein, a “therapeutically effective amount” of a compound with respect to the subject method of treatment, refers to an amount of the compound(s) in a preparation which, when administered as part of a desired dosage regimen (to a patient, e.g., a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment. As used herein, the term “treating” or “treatment” includes reversing, reducing, or arresting the symptoms, clinical signs, and underlying pathology of a condition in manner to improve or stabilize a patient's condition.

The LMP2-selective inhibitor and the LMP7-selective inhibitor can be administered simultaneously or sequentially. In some cases, the LMP2-selective and the LMP7-selective inhibitors are administered simultaneously. When the LMP2-selective and the LMP7-selective inhibitors are administered simultaneously, the two compounds can either be co-formulated and administered in the same composition, or formulated and administered in separate compositions. In cases when the LMP2-selective and the LMP7-selective inhibitors are administered sequentially, the inhibitors can be administered in any order. In some cases, the LMP2-selective inhibitor is administered before the LMP7-selective inhibitor. In various cases, the LMP7-selective inhibitor is administered before the LMP2-selective inhibitor. When the LMP2-selective and the LMP7-selective inhibitors are administered either simultaneously in separate compositions or sequentially, they can be administered to the patient at the same site or at different sites (e.g., at the same injection site if subcutaneous administration, or at different injection sites). In embodiments when the LMP2-selective inhibitor and LMP7-selective inhibitors are administered sequentially, they can be administered within 24 hours of each other. In various cases, the LMP2-inhibitor and LMP7-inhibitors are administred within 21 hours, 18 hours, 15 hours, 12 hours, 9, hours, 6 hours, 3 hours, 2 hours, 1 hour, 45 minutes, 30 minutes, 15 minutes, 10 minutes, 5 minutes, or 1 minute of each other.

As described in further detail below, the LMP2-selective and LMP7-selective inhibitors can be administered orally or parenterally (e.g., intravenously or subcutaneously). In some specific cases, the LMP2-selective inhibitor and/or the LMP7-selective inhibitor are administered subcutaneously.

The LMP2-selective inhibitor and/or the LMP7-selective inhibitor can be present as a pharmaceutically acceptable salt thereof. The term “pharmaceutically acceptable salt” refers to the relatively non-toxic, inorganic and organic acid addition salts of a compound provided herein. These salts can be prepared in situ during the final isolation and purification of a compound provided herein, or by separately reacting the compound in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

In some embodiments, a compound provided herein may contain one or more acidic functional groups and, thus, is capable of forming pharmaceutically acceptable salts with pharmaceutically acceptable bases. The term “pharmaceutically acceptable salts” in these instances refers to the relatively non-toxic inorganic and organic base addition salts of a compound provided herein. These salts can likewise be prepared in situ during the final isolation and purification of the compound, or by separately reacting the purified compound in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

### Immune-Related Disorders

The methods provided herein are useful in the treatment of immune-related disorders. An “immune-related dislorder” as used herein is a disease or disorder arising from and directed against an individual's own tissues. Examples of immune-related disorders include, but are not limited to, inflammatory responses such as inflammatory skin diseases including psoriasis and dermatitis (e.g., atopic dermatitis); systemic scleroderma and sclerosis; responses associated with inflammatory bowel disease (such as Crohn's disease and ulcerative colitis); respiratory distress syndrome (including adult respiratory distress syndrome(ARDS)); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g., Type I diabetes mellitus or insulin dependent diabetes mellitus); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Beheet disease; giant cell arteritis; immune complex nephritis; IgA nephropathy; IgM polyneuropathies; immune thrombocytopenic purpura (ITP) or autoimmune thrombocytopenia. In specific cases, the autoimmune disease is systemic lupus erythematosus or lupus nephritis. In some cases, the autoimmune disease is systemic vasculitis or idiopathic inflammatory myopathy.

In some embodiments, the immune-related disorder is selected from the group consisting of rheumatoid arthritis, lupus, multiple sclerosis, psoriasis, chronic obstructive pulmonary disease (“COPD”), granulomatosis and vasculitis, graft or transplant-related disease, and fibrotic disease. In various cases, the immune-related disorder is rheumatoid arthritis, lupus, multiple sclerosis, psoriasis, COPD, or granulomatosis and vasculitis. In cases when the immune-related disorder is lupus, the lupus can be systemic lupus erythematosus (“SLE”), subacute cutaneous lupus, drug-induced lupus, neonatal lupus, or discoid lupus. In cases when the immune-related disorder is a fibrotic disease, the fibrotic disease can be selected from the group consisting of cystic fibrosis, idiopathic pulmonary fibrosis (“IPF”), cirrhosis, biliary atresia, atrial fibrosis, endomyocardial fibrosis, arterial stiffness, arthrofibrosis, Crohn's Disease, Dupuytren's contracture, keloid, mediastinal fibrosis, myelofibrosis, Peyronie's disease, nephrogenic systemic fibrosis, progressive massive fibrosis, retroperitoneal fibrosis, and scleroderma.

### LMP2-Selective Inhibitors

The LMP2-selective inhibitors described herein induce selective inhibition of the LMP2 subunit over the LMP7 subunit of the immunoproteasome. The LMP2 subunit of the immunoproteasome has been implicated in regulating cell growth of various tumors, and may be implicated in prostate cancer. See Wehenkel et al., Brit. J. Cancer, 107:53-62 (2012) and Ho et al., Chem. & Biol., 14:419-430 (2007).

In some cases, the LMP2-selective inhibitors have an IC₅₀ for the LMP2 subunit of about 1 nm to about 1 μM, as measured by active site ELISA, and in some cases, induce at least at least 2-fold, or at least 8.5-fold inhibition of the LMP2 subunit over the LMP7 subunit, as measured by active site ELISA. Active site ELISA measurement for activity of LMP2 and LMP7 is well known in the art, e.g., as discussed in Parlati et al, Blood, 114(16):3439-47 (2009). Inhibition of LMP2 and LMP7 by a compound as disclosed herein can alternatively be measured by any known assay. In some embodiments, the LMP2-selective inhibitors disclosed herein have an IC₅₀ for the LMP2 subunit of about 1 nm up to about 2 nm, or 3 nm, or 4 nm, or 5 nm, or 6 nm, or 7 nm, or 8 nm, or 9 nm, or 10 nm, or 15 nm, or 20 nm, or 25 nm, or 30 nm, or 40 nm, or 50 nm, or 60 nm, or 70 nm, or 80 nm, or 90 nm, or 100 nm, or 150 nm, or 200 nm, or 250 nm, 300 nm, or about 350 nm, or about 400 nm, or about 450 nm, or about 500 nm, or about 550 nm, or about 600 nm, or about 700 nm, or about 800 nm, or about 900 nm. In some cases, the LMP2-selective inhibitor inhibits the LMP2 subunit over the LMP7 subunit by at least 2-fold, or 3-fold, or 4-fold, or 5-fold, or 6-fold, or 7-fold, or 8-fold. In some cases, the LMP2-selective inhibitor inhibitors the LMP2 subunit over the LMP7 subunit by at least 8.5-fold, or 9-fold, or 9.5-fold, or 10-fold, or 11-fold, or 12-fold, or 13-fold, or 14-fold, or 15-fold, or 16-fold, or 17-fold, or 18-fold, or 19-fold, or 20-fold, or 25-fold, or 30-fold, or 40-fold, or 50-fold. In some cases, the LMP2-selective inhibitor can inhibit LMP2 activity by at least 10%, at least 20%, at least 30%, at least 40%, at least 50%, at least 60%, at least 70%, or at least 80%, e.g., as measured by active site ELISA.

Contemplated LMP2-selective inhibitors include those described in WO 2014/152127, which is incorporated by reference in its entirety. Some specific LMP2-selective inhibitors contemplated include those having a structure of Formula (II), or a pharmaceutically acceptable salt thereof:

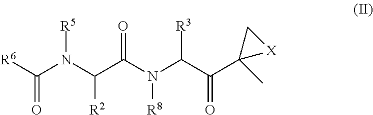

wherein:

X is selected from O, S, NH, and N—C₁₋₆alkyl;

R² and R³ are each independently selected from aryl, C₁₋₆aralkyl, heteroaryl, and _{c1-6}heteroaralkyl;

R⁵ is selected from hydrogen, OH, C₁₋₆aralkyl, and C₁₋₆alkyl;

R⁶ is heteroaryl, piperidinyl, piperazinyl, morpholinyl, a lactone, a lactam, or

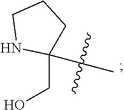 [SMILES: *C1(CO)CCCN1]

and

R⁸ is selected from hydrogen, C₁₋₆alkyl, and C₁₋₆aralkyl. Other LMP2-selective inhibitors include those as disclosed in, e.g., de Bruin, et al, J. Med. Chem. 57:6197-6209 (2014) and Ho, et al, Chemistry & Biology, 14: 419-430 (2007).

In some embodiments, the LMP2-selective inhibitor can have a structure selected from the group consisting of:

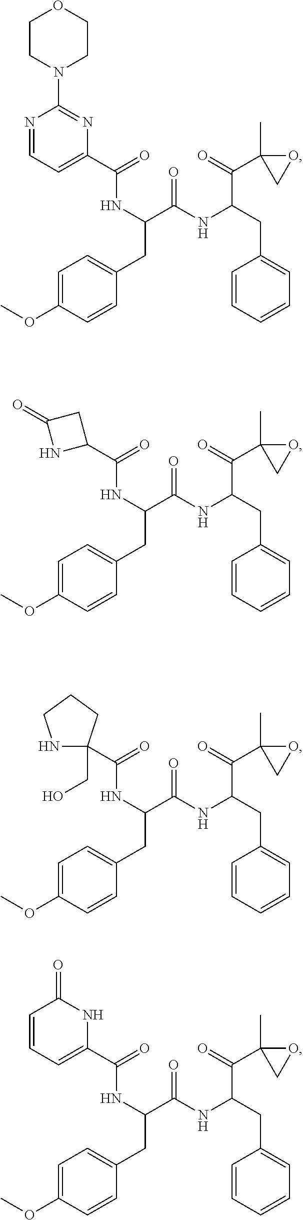 [SMILES: COc1ccc(CC(NC(=O)c2ccnc(N3CCOCC3)n2)C(=O)NC(Cc2ccccc2)C(=O)C2(C)CO2)cc1 ; COc1ccc(CC(NC(=O)C2CC(=O)N2)C(=O)NC(Cc2ccccc2)C(=O)C2(C)CO2)cc1 ; COc1ccc(CC(NC(=O)C2(CO)CCCN2)C(=O)NC(Cc2ccccc2)C(=O)C2(C)CO2)cc1 ; COc1ccc(CC(NC(=O)c2cccc(=O)[nH]2)C(=O)NC(Cc2ccccc2)C(=O)C2(C)CO2)cc1]

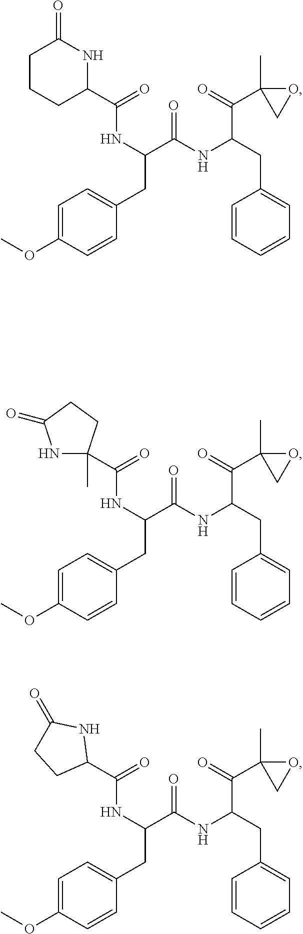 [SMILES: COc1ccc(CC(NC(=O)C2CCCC(=O)N2)C(=O)NC(Cc2ccccc2)C(=O)C2(C)CO2)cc1 ; COc1ccc(CC(NC(=O)C2(C)CCC(=O)N2)C(=O)NC(Cc2ccccc2)C(=O)C2(C)CO2)cc1 ; COc1ccc(CC(NC(=O)C2CCC(=O)N2)C(=O)NC(Cc2ccccc2)C(=O)C2(C)CO2)cc1]

and a combination thereof, or a pharmaceutically acceptable salt thereof. In more specific embodiments, the LMP2-selective can have a structure selected from the group consisting of:

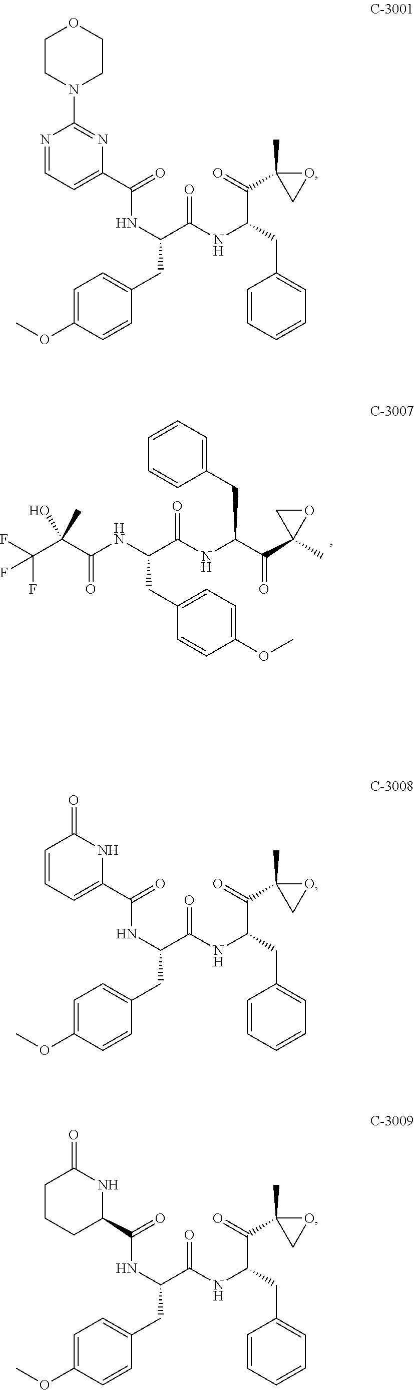

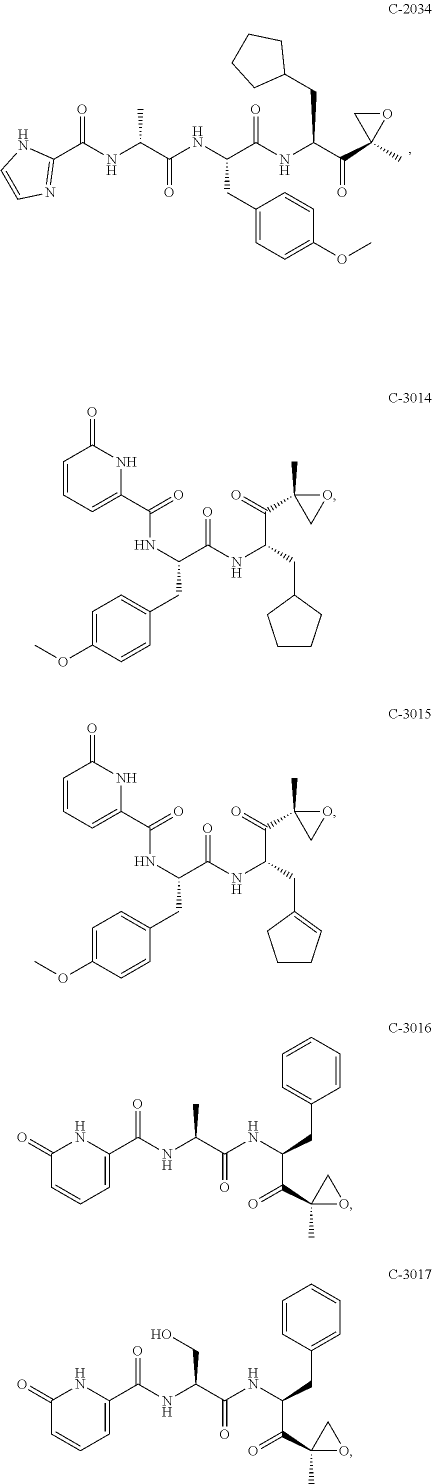

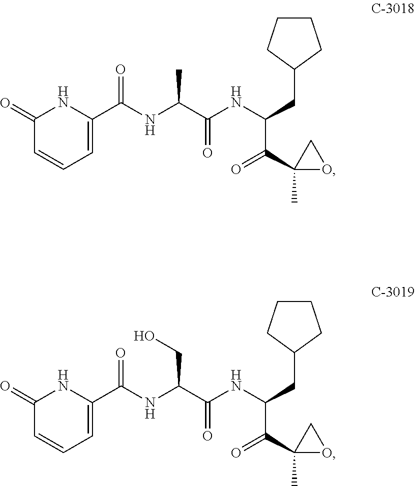 [SMILES: C[C@H](NC(=O)c1cccc(=O)[nH]1)C(=O)N[C@@H](CC1CCCC1)C(=O)[C@@]1(C)CO1 ; C[C@]1(C(=O)[C@H](CC2CCCC2)NC(=O)[C@H](CO)NC(=O)c2cccc(=O)[nH]2)CO1]

and a combination thereof, or a pharmaceutically acceptable salt thereof. In some cases, the LMP2-selective inhibitor is

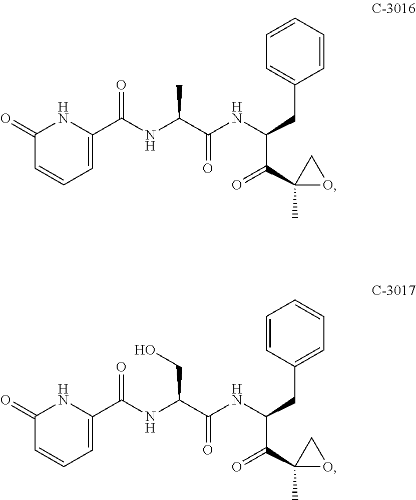 [SMILES: C[C@H](NC(=O)c1cccc(=O)[nH]1)C(=O)N[C@@H](Cc1ccccc1)C(=O)[C@@]1(C)CO1 ; C[C@]1(C(=O)[C@H](Cc2ccccc2)NC(=O)[C@H](CO)NC(=O)c2cccc(=O)[nH]2)CO1]

or a combination thereof, or a pharmaceutically acceptable salt thereof.

The LMP2-selective inhibitors can be administered once weekly (e.g., every seven days) up to once bi-monthly (e.g., every 15 days), e.g., once every 7 days, once every 8 days, once every 9 days, once every 10 days, once every 11 days, once every 12 days, once every 13 days, once every 14 days, or once every 15 days. The dose of the LMP2-selective inhibitor can be 1 to 300 mg/day. If the dose frequency is less than once daily (e.g., every 7 days), the total dose given to the subject will be multiplied by that amount e.g., 7 to 2100 mg given once every 7 days. In some cases, the LMP2-selective inhibitor dose is 40 to 120 mg/day (which can also be given in less than daily dosing frequency). Thus, the daily dose of the LMP2-selective inhibitor does not indicate that the amount is given daily, but could be combined with other daily doses to be administered to the subject in less frequent doses.

### LMP7-Selective Inhibitors

The LMP7-selective inhibitors described herein induce selective inhibition of the LMP7 subunit over the LMP2 subunit of the immunoproteasome. The LMP7 subunit of the immunoproteasome has been implicated in reduction of cytokine activity or expression, e.g., one or more of IL-2, MHC-I, IL-6, TNFα, and IFN-γ.

In some cases, the LMP7-selective inhibitors have an IC₅₀ for the LMP7 subunit of about 1 nm to about 1 μM, as measured by active site ELISA and, in some cases, can induce at least 4-fold inhibition of the LMP7 subunit over the LMP2 subunit, as measured by active site ELISA. Active site ELISA measurement for activity of LMP2 and LMP7 is well known in the art, e.g., as discussed in Parlati et al, Blood, 114(16):3439-47 (2009). Inhibition of LMP2 and LMP7 by a compound as disclosed herein can alternatively be measured by any known assay. In some embodiments, the LMP7-selective inhibitors disclosed herein have an IC₅₀ for the LMP7 subunit of about 1 nm up to about 2 nm, or 3 nm, or 4 nm, or 5 nm, or 6 nm, or 7 nm, or 8 nm, or 9 nm, or 10 nm, or 15 nm, or 20 nm, or 25 nm, or 30 nm, or 40 nm, or 50 nm, or 60 nm, or 70 nm, or 80 nm, or 90 nm, or 100 nm, or 150 nm, or 200 nm, or 250 nm, 300 nm, or about 350 nm, or about 400 nm, or about 450 nm, or about 500 nm, or about 550 nm, or about 600 nm, or about 700 nm, or about 800 nm, or about 900 nm. In some cases, the LMP7-selective inhibitor inhibits the LMP7 subunit over the LMP2 subunit by at least 2-fold, or 3-fold. In various cases, the LMP7-selective inhibitor inhibits the LMP7 subunit over the LMP2 subunit by at least 4-fold, or 5-fold, or 6-fold, or 7-fold, or 8-fold, or 9-fold, or 10-fold, or 11-fold, or 12-fold, or 13-fold, or 14-fold, or 15-fold, or 16-fold, or 17-fold, or 18-fold, or 19-fold, or 20-fold, or 25-fold, or 30-fold, or 40-fold, or 50-fold . In some cases, the LMP7-selective inhibitor can inhibit LMP7 activity by at least 10%, at least 20%, at least 30%, at least 40%, at least 50%, at least 60%, at least 70%, or at least 80%, as measured by active site ELISA.

Contemplated LMP7-selective inhibitors include those described in WO 2014/152134, which is incorporated by reference in its entirety. Some specific LMP7-selective inhibitors contemplated include those having a structure of Formula (X), or a pharmaceutically acceptable salt thereof:

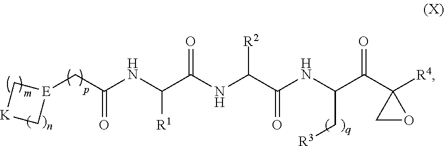

wherein:

m and n each independently are 0, 1 or 2, and m +n =2, 3, or 4;

p is 0 or 1;

q is 0, 1, or 2;

K is selected from the group consisting of CR⁵R⁶, NR⁷, N(C═O)OR⁷, —NH—(C═O)—,

O, S, SO, and SO₂;

E is N or CR⁷;

R¹ is selected from the group consisting of H, C₁₋₆alkyl, C₂₋₆alkenyl, C₂₋₆alkynyl, C₃₋₆cycloalkyl, and 3-6 membered heterocycloalkyl, wherein R¹ is optionally substituted with one or more substituents selected from the group consisting of halo, OR⁷, SR⁷, N(R⁷)₂, CN, and (C═O)N(R⁷)₂;

R² is C₁₋₂alkylene-G or (C═O)-G; wherein G is selected from the group consisting of aryl, heteroaryl, and pyridinone, with the proviso that when R² is CH₂phenyl, the phenyl is substituted with one or more substituents selected from the group consisting of OR⁷, halo, C₁₋₃alkyl, OCF₃, SO₂R⁷, (C═O)N(R⁷)₂, CN, and SO₂N(R⁷)₂;

R³ is non-aromatic and selected from the group consisting of C₃₋₇cycloalkyl, C₃₋₇cycloalkenyl, a 3-7 membered heterocycloalkyl, and a 3-7 membered heterocycloalkenyl, wherein R³ is optionally substituted with one or more substituents selected from the group consisting of halo, ═O, OR⁷, SR⁷, N(R⁷)₂, O(C═O)N(R⁷)₂, and C₁₋₆alkyl;

R⁴ is H or C₁₋₃alkyl;

R⁵ and R⁶ are each independently selected from the group consisting of H, OH, halo, C₁₋₃alkyl, and CF₃, or R⁵ and R⁶ together with the carbon to which they are attached form C═O or

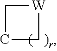

wherein W is O or NR⁷, and r is 1, 2 or 3; and each R⁷ is independently H or C₁₋₆alkyl. Other LMP7-selective inhibitors include those discloses in WO 2015/195950, US 20150299143, WO 2016/050356, WO 2014/056748 A1, and WO 2014/056954 A1, each of which is incorporated by reference in its entirety, Sosič, et al, Angew. Chem. Int. Ed., 55:1-5 (2016); de Bruin, et al, J. Med. Chem. 57:6197-6209 (2014); and Dubiella, et al, Angew. Chem. Int. Ed. 54:15888-15891 (2015).

In some cases, the LMP7-selective inhibitor can have a structure selected from group consisting of:

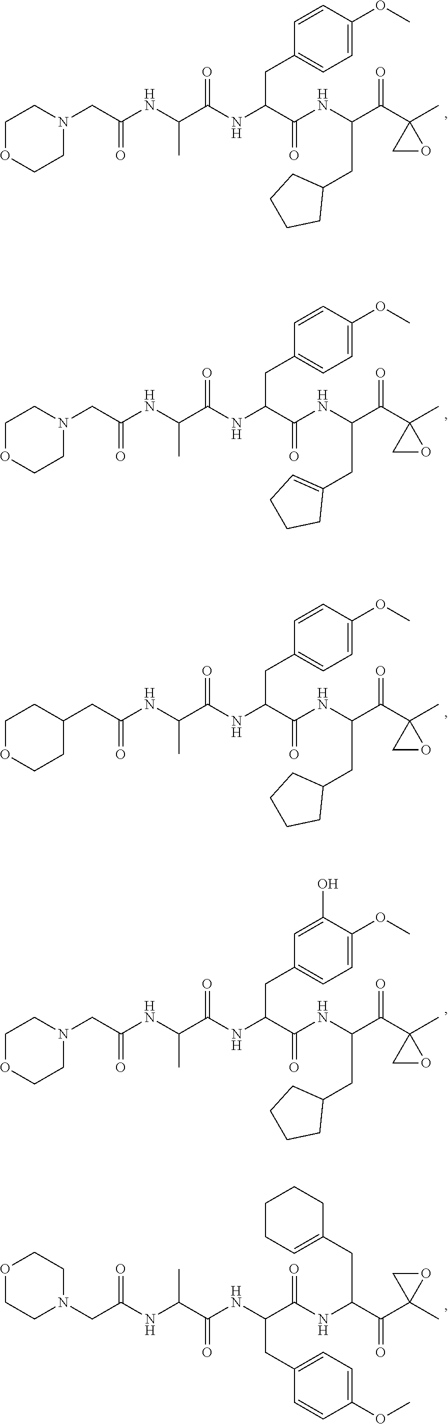 [SMILES: COc1ccc(CC(NC(=O)C(C)NC(=O)CN2CCOCC2)C(=O)NC(CC2CCCC2)C(=O)C2(C)CO2)cc1 ; COc1ccc(CC(NC(=O)C(C)NC(=O)CN2CCOCC2)C(=O)NC(CC2=CCCC2)C(=O)C2(C)CO2)cc1 ; COc1ccc(CC(NC(=O)C(C)NC(=O)CC2CCOCC2)C(=O)NC(CC2CCCC2)C(=O)C2(C)CO2)cc1 ; COc1ccc(CC(NC(=O)C(C)NC(=O)CN2CCOCC2)C(=O)NC(CC2CCCC2)C(=O)C2(C)CO2)cc1O ; COc1ccc(CC(NC(=O)C(C)NC(=O)CN2CCOCC2)C(=O)NC(CC2=CCCCC2)C(=O)C2(C)CO2)cc1]

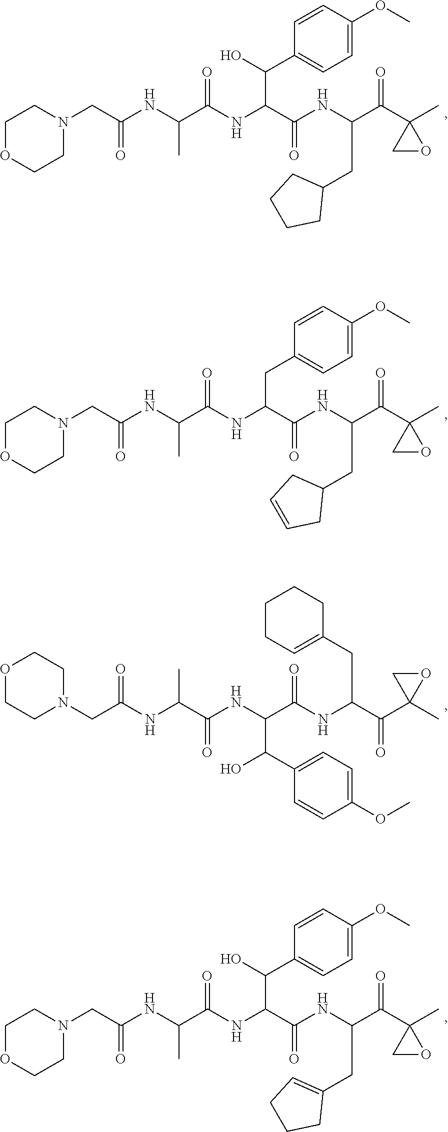 [SMILES: COc1ccc(C(O)C(NC(=O)C(C)NC(=O)CN2CCOCC2)C(=O)NC(CC2CCCC2)C(=O)C2(C)CO2)cc1 ; COc1ccc(CC(NC(=O)C(C)NC(=O)CN2CCOCC2)C(=O)NC(CC2CC=CC2)C(=O)C2(C)CO2)cc1 ; COc1ccc(C(O)C(NC(=O)C(C)NC(=O)CN2CCOCC2)C(=O)NC(CC2=CCCCC2)C(=O)C2(C)CO2)cc1 ; COc1ccc(C(O)C(NC(=O)C(C)NC(=O)CN2CCOCC2)C(=O)NC(CC2=CCCC2)C(=O)C2(C)CO2)cc1]

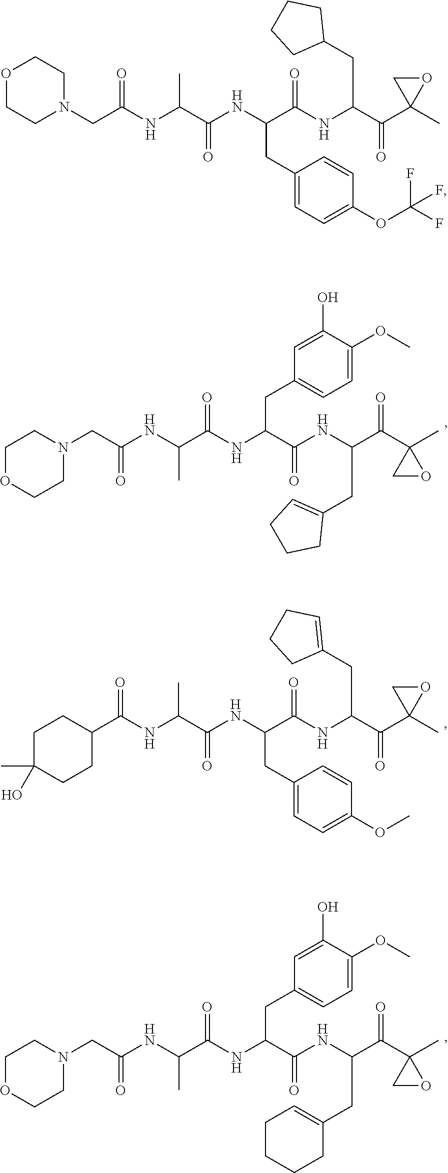

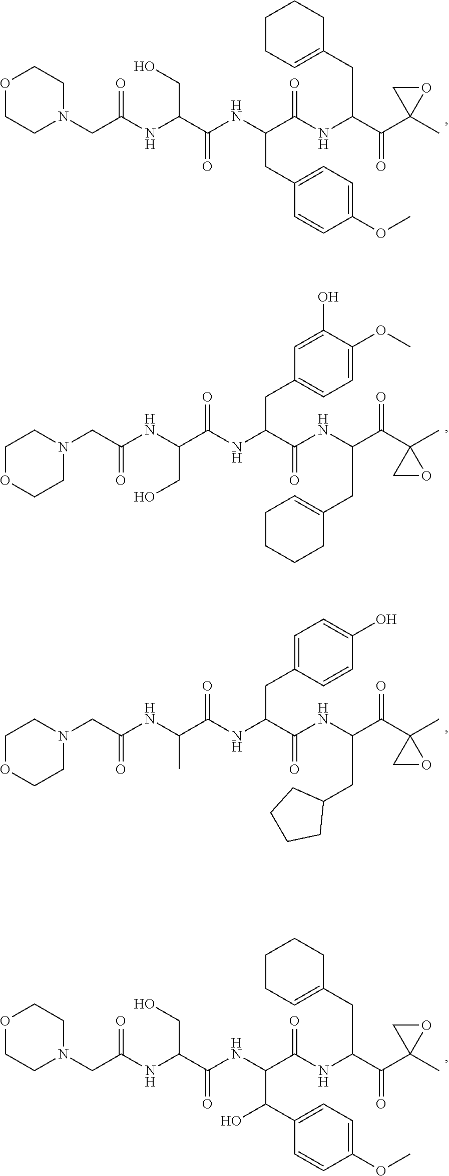 [SMILES: COc1ccc(CC(NC(=O)C(CO)NC(=O)CN2CCOCC2)C(=O)NC(CC2=CCCCC2)C(=O)C2(C)CO2)cc1 ; COc1ccc(CC(NC(=O)C(CO)NC(=O)CN2CCOCC2)C(=O)NC(CC2=CCCCC2)C(=O)C2(C)CO2)cc1O ; CC(NC(=O)CN1CCOCC1)C(=O)NC(Cc1ccc(O)cc1)C(=O)NC(CC1CCCC1)C(=O)C1(C)CO1 ; COc1ccc(C(O)C(NC(=O)C(CO)NC(=O)CN2CCOCC2)C(=O)NC(CC2=CCCCC2)C(=O)C2(C)CO2)cc1]

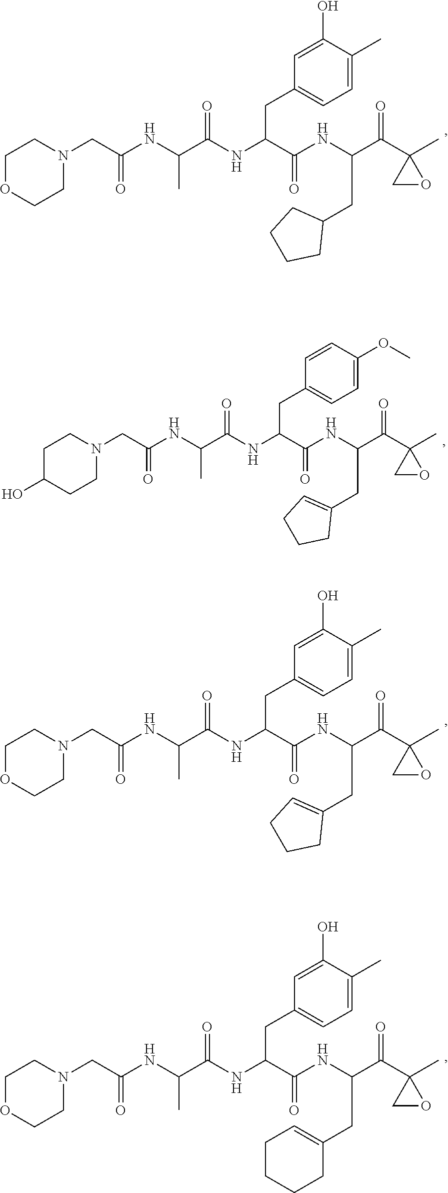 [SMILES: Cc1ccc(CC(NC(=O)C(C)NC(=O)CN2CCOCC2)C(=O)NC(CC2CCCC2)C(=O)C2(C)CO2)cc1O ; COc1ccc(CC(NC(=O)C(C)NC(=O)CN2CCC(O)CC2)C(=O)NC(CC2=CCCC2)C(=O)C2(C)CO2)cc1 ; Cc1ccc(CC(NC(=O)C(C)NC(=O)CN2CCOCC2)C(=O)NC(CC2=CCCC2)C(=O)C2(C)CO2)cc1O ; Cc1ccc(CC(NC(=O)C(C)NC(=O)CN2CCOCC2)C(=O)NC(CC2=CCCCC2)C(=O)C2(C)CO2)cc1O]

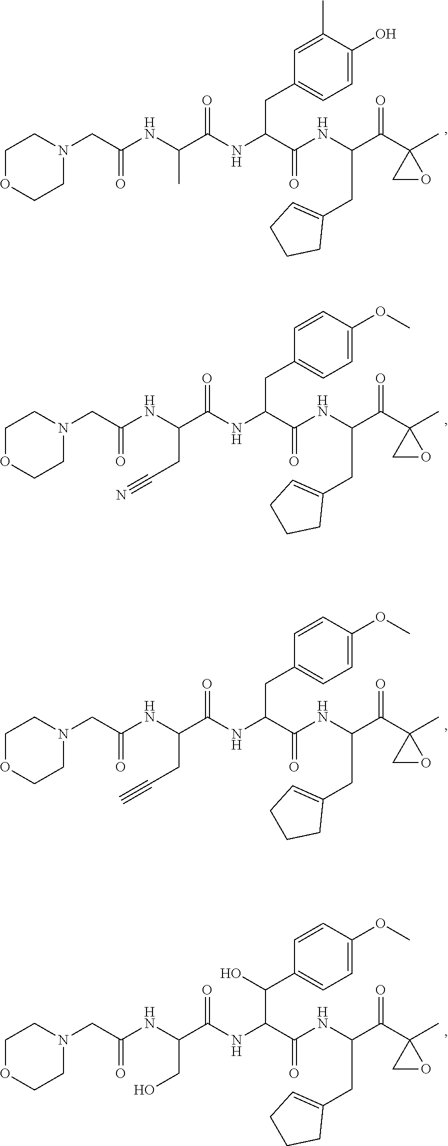 [SMILES: Cc1cc(CC(NC(=O)C(C)NC(=O)CN2CCOCC2)C(=O)NC(CC2=CCCC2)C(=O)C2(C)CO2)ccc1O ; COc1ccc(CC(NC(=O)C(CC#N)NC(=O)CN2CCOCC2)C(=O)NC(CC2=CCCC2)C(=O)C2(C)CO2)cc1 ; C#CCC(NC(=O)CN1CCOCC1)C(=O)NC(Cc1ccc(OC)cc1)C(=O)NC(CC1=CCCC1)C(=O)C1(C)CO1 ; COc1ccc(C(O)C(NC(=O)C(CO)NC(=O)CN2CCOCC2)C(=O)NC(CC2=CCCC2)C(=O)C2(C)CO2)cc1]

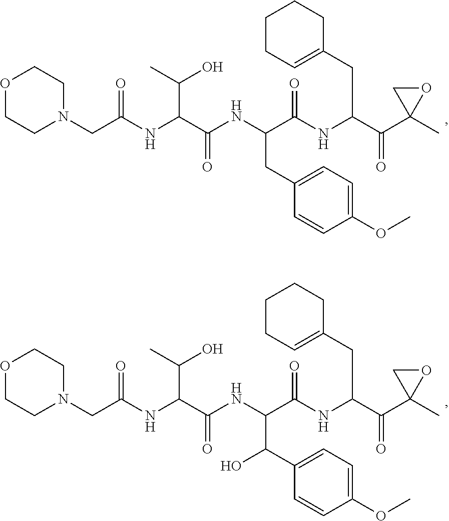 [SMILES: COc1ccc(CC(NC(=O)C(NC(=O)CN2CCOCC2)C(C)O)C(=O)NC(CC2=CCCCC2)C(=O)C2(C)CO2)cc1 ; COc1ccc(C(O)C(NC(=O)C(NC(=O)CN2CCOCC2)C(C)O)C(=O)NC(CC2=CCCCC2)C(=O)C2(C)CO2)cc1]

and a combination thereof, or a pharmaceutically acceptable salt thereof. In more specific embodiments, the LMP7-selective inhibitor is selected from the group consisting of:

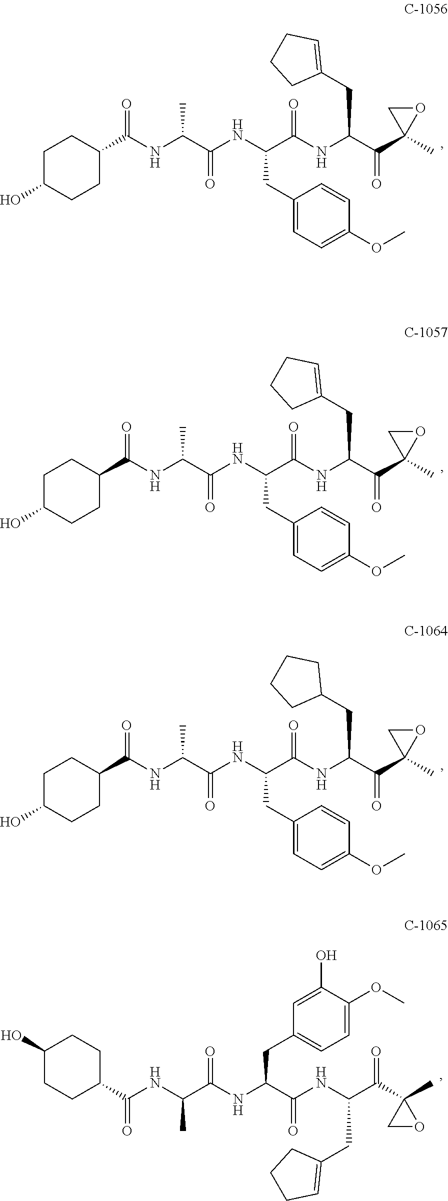 [SMILES: COc1ccc(C[C@H](NC(=O)[C@@H](C)NC(=O)[C@H]2CC[C@@H](O)CC2)C(=O)N[C@@H](CC2=CCCC2)C(=O)[C@@]2(C)CO2)cc1 ; COc1ccc(C[C@H](NC(=O)[C@@H](C)NC(=O)[C@H]2CC[C@H](O)CC2)C(=O)N[C@@H](CC2=CCCC2)C(=O)[C@@]2(C)CO2)cc1 ; COc1ccc(C[C@H](NC(=O)[C@@H](C)NC(=O)[C@H]2CC[C@H](O)CC2)C(=O)N[C@@H](CC2CCCC2)C(=O)[C@@]2(C)CO2)cc1 ; COc1ccc(C[C@H](NC(=O)[C@@H](C)NC(=O)[C@H]2CC[C@H](O)CC2)C(=O)N[C@@H](CC2=CCCC2)C(=O)[C@@]2(C)CO2)cc1O]

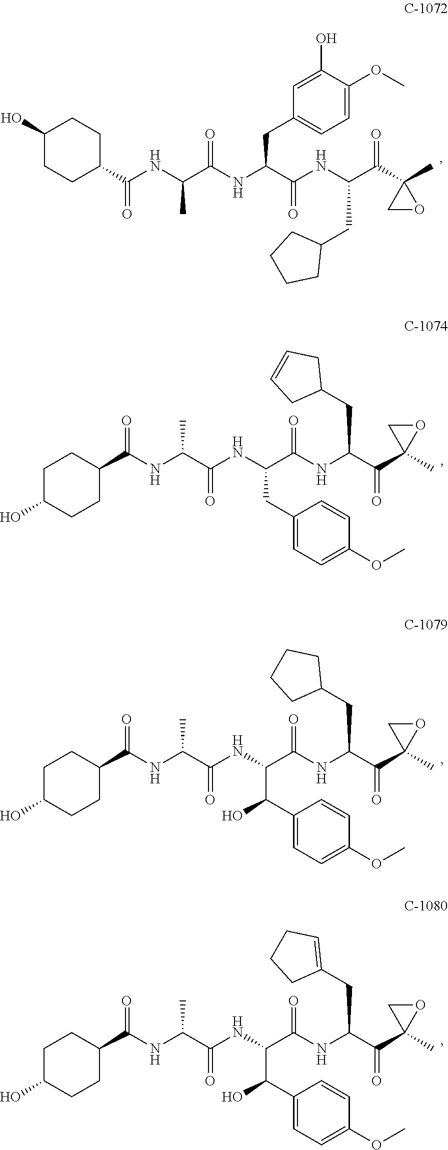 [SMILES: COc1ccc(C[C@H](NC(=O)[C@@H](C)NC(=O)[C@H]2CC[C@H](O)CC2)C(=O)N[C@@H](CC2CCCC2)C(=O)[C@@]2(C)CO2)cc1O ; COc1ccc(C[C@H](NC(=O)[C@@H](C)NC(=O)[C@H]2CC[C@H](O)CC2)C(=O)N[C@@H](CC2CC=CC2)C(=O)[C@@]2(C)CO2)cc1 ; COc1ccc([C@@H](O)[C@H](NC(=O)[C@@H](C)NC(=O)[C@H]2CC[C@H](O)CC2)C(=O)N[C@@H](CC2CCCC2)C(=O)[C@@]2(C)CO2)cc1 ; COc1ccc([C@@H](O)[C@H](NC(=O)[C@@H](C)NC(=O)[C@H]2CC[C@H](O)CC2)C(=O)N[C@@H](CC2=CCCC2)C(=O)[C@@]2(C)CO2)cc1]

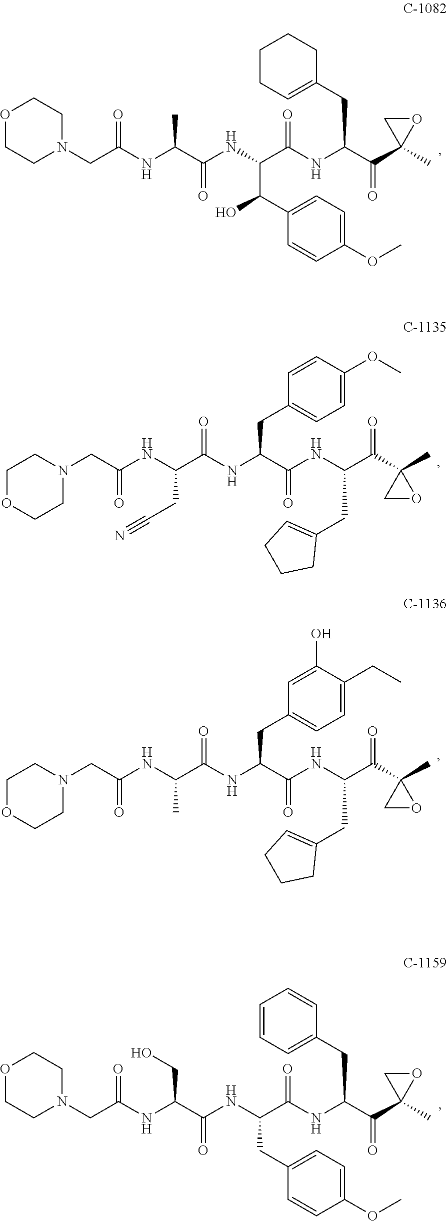 [SMILES: COc1ccc([C@@H](O)[C@H](NC(=O)[C@H](C)NC(=O)CN2CCOCC2)C(=O)N[C@@H](CC2=CCCCC2)C(=O)[C@@]2(C)CO2)cc1 ; COc1ccc(C[C@H](NC(=O)[C@H](CC#N)NC(=O)CN2CCOCC2)C(=O)N[C@@H](CC2=CCCC2)C(=O)[C@@]2(C)CO2)cc1 ; CCc1ccc(C[C@H](NC(=O)[C@H](C)NC(=O)CN2CCOCC2)C(=O)N[C@@H](CC2=CCCC2)C(=O)[C@@]2(C)CO2)cc1O ; COc1ccc(C[C@H](NC(=O)[C@H](CO)NC(=O)CN2CCOCC2)C(=O)N[C@@H](Cc2ccccc2)C(=O)[C@@]2(C)CO2)cc1]

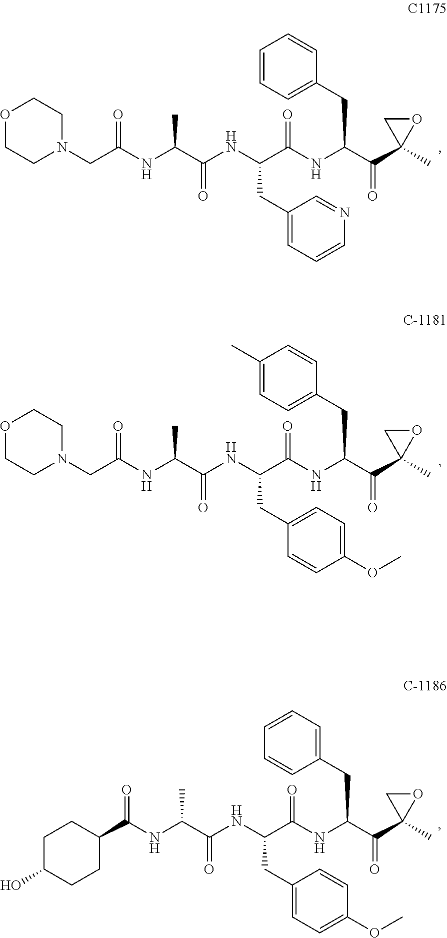 [SMILES: C[C@H](NC(=O)CN1CCOCC1)C(=O)N[C@@H](Cc1cccnc1)C(=O)N[C@@H](Cc1ccccc1)C(=O)[C@@]1(C)CO1 ; COc1ccc(C[C@H](NC(=O)[C@H](C)NC(=O)CN2CCOCC2)C(=O)N[C@@H](Cc2ccc(C)cc2)C(=O)[C@@]2(C)CO2)cc1 ; COc1ccc(C[C@H](NC(=O)[C@@H](C)NC(=O)[C@H]2CC[C@H](O)CC2)C(=O)N[C@@H](Cc2ccccc2)C(=O)[C@@]2(C)CO2)cc1]

and a combination thereof, or a pharmaceutically acceptable salt thereof. In some cases, the LMP7-selective inhibitor is selected from the group consisting of C-1056, C-1057, C-1064, C-1065, C-1072, C-1074, C-1079, C-1080, C-1186, and a combination thereof, or a pharmaceutically acceptable salt thereof.

The LMP7-selective inhibitors can be administered once weekly (e.g., every seven days) up to once bi-monthly (e.g., every 15 days), e.g., once every 7 days, once every 8 days, once every 9 days, once every 10 days, once every 11 days, once every 12 days, once every 13 days, once every 14 days, or once every 15 days. The dose of the LMP7-selective inhibitor can be 1 to 300 mg/day. If the dose frequency is less than once daily (e.g., every 7 days), the total dose given to the subject will be multiplied by that amount e.g., 7 to 2100 mg given once every 7 days. In some cases, the LMP7-selective inhibitor dose is 40 to 120 mg/day (which can also be given in less than daily dosing frequency). Thus, the daily dose of the LMP7-selective inhibitor does not indicate that the amount is given daily, but could be combined with other daily doses to be administered to the subject in less frequent doses.

### Combinations

Contemplated combination therapies include administration of a LMP2-selective inhibitor described in WO 2014/152127 and a LMP7-selective inhibitor described in WO 2014/152134. Specifically contemplated combination therapies include administration of a LMP2-selective inhibitor having a structure Formula (II), or a pharmaceutically acceptable salt thereof, and a LMP7-selective inhibitor having a structure of Formula (X), or a pharmaceutically acceptable salt thereof, each as previously described herein. In some cases, the combination therapy described herein includes a LMP2-selective inhibitor selected from the group consisting of C-2034, C-3001, C-3007, C-3008, C-3009, C-3014, C-3015, C-3016, C-3017, C-3018, and C-3019, and a LMP7-selective inhibitor selected from the group consisting of C-1056, C-1057, C-1064, C-1065, C-1072, C-1074, C-1075, C-1079, C-1080, C-1082, C-1135, C-1136, C-1159, C-1175, C-1181, and C-1186, or a pharmaceutically acceptable salt of any of the foregoing. In some specific embodiments, the combination therapy described herein includes a LMP2-selective inhibitor selected from the group consisting of C-3016 and C-3017, and a LMP7-selective inhibitor selected from the group consisting of C-1056, C-1057, C-1064, C-1065, C-1072, C-1074, C-1079, C-1080, and C-1186, or a pharmaceutically acceptable salt of any of the foregoing.

In some embodiments, the LMP2-selective inhibitor is C-3016 and the LMP7-selective inhibitor is C-1056, or pharmaceutically acceptable salts thereof. In some embodiments, the LMP2-selective inhibitor is C-3016 and the LMP7-selective inhibitor is C-1057, or pharmaceutically acceptable salts thereof. In some embodiments, the LMP2-selective inhibitor is C-3016 and the LMP7-selective inhibitor is C-1064, or pharmaceutically acceptable salts thereof. In some embodiments, the LMP2-selective inhibitor is C-3016 and the LMP7-selective inhibitor is C-1065, or pharmaceutically acceptable salts thereof. In some embodiments, the LMP2-selective inhibitor is C-3016 and the LMP7-selective inhibitor is C-1072, or pharmaceutically acceptable salts thereof. In some embodiments, the LMP2-selective inhibitor is C-3016 and the LMP7-selective inhibitor is C-1074, or pharmaceutically acceptable salts thereof. In some embodiments, the LMP2-selective inhibitor is C-3016 and the LMP7-selective inhibitor is C-1079, or pharmaceutically acceptable salts thereof. In some embodiments, the LMP2-selective inhibitor is C-3016 and the LMP7-selective inhibitor is C-1080, or pharmaceutically acceptable salts thereof. In some embodiments, the LMP2-selective inhibitor is C-3016 and the LMP7-selective inhibitor is C-1186, or pharmaceutically acceptable salts thereof.

In some embodiments, the LMP2-selective inhibitor is C-3017 and the LMP7-selective inhibitor is C-1056, or pharmaceutically acceptable salts thereof. In some embodiments, the LMP2-selective inhibitor is C-3017 and the LMP7-selective inhibitor is C-1057, or pharmaceutically acceptable salts thereof. In some embodiments, the LMP2-selective inhibitor is C-3017 and the LMP7-selective inhibitor is C-1064, or pharmaceutically acceptable salts thereof. In some embodiments, the LMP2-selective inhibitor is C-3017 and the LMP7-selective inhibitor is C-1065, or pharmaceutically acceptable salts thereof. In some embodiments, the LMP2-selective inhibitor is C-3017 and the LMP7-selective inhibitor is C-1072, or pharmaceutically acceptable salts thereof. In some embodiments, the LMP2-selective inhibitor is C-3017 and the LMP7-selective inhibitor is C-1074, or pharmaceutically acceptable salts thereof. In some embodiments, the LMP2-selective inhibitor is C-3017 and the LMP7-selective inhibitor is C-1079, or pharmaceutically acceptable salts thereof. In some embodiments, the LMP2-selective inhibitor is C-3017 and the LMP7-selective inhibitor is C-1080, or pharmaceutically acceptable salts thereof. In some embodiments, the LMP2-selective inhibitor is C-3017 and the LMP7-selective inhibitor is C-1186, or pharmaceutically acceptable salts thereof.

### Pharmaceutical Formulations and Administration

The LMP2-selective and LMP7-selective inhibitors described herein can be formulated and administered in the same (co-formulated) or different pharmaceutical formulations. Thus, the disclosure also provides a pharmaceutical composition comprising a LMP2-selective inhibitor and/or a LMP7-selective inhibitor; and a pharmaceutically acceptable carrier. Specifically, the disclosure provides a pharmaceutical composition comprising a LMP2-selective inhibitor, a LMP7-selective inhibitor, and a pharmaceutically acceptable carrier

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition, or vehicle, such as a liquid or solid filler, diluent, excipient, solvent or encapsulating material. As used herein the language “pharmaceutically acceptable carrier” includes buffer, sterile water for injection, solvents, dispersion media, coatings, antibacterial and antifungal agents, isotonic and absorption delaying agents, and the like, compatible with pharmaceutical administration. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions provided herein are non-pyrogenic, i.e., do not induce significant temperature elevations when administered to a patient.

In some embodiments, the pharmaceutically acceptable carrier comprises a cyclodextrin. Suitable cyclodextrins include, for example, a beta-cyclodextrin, e.g., sulfobutylether-beta-cyclodextrin, (2-hydroxypropyl)-beta-cyclodextrin, and combinations thereof.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

A pharmaceutical composition may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars and the like into the compositions. In addition, prolonged absorption of the injectable pharmaceutical form may be brought about by the inclusion of agents which delay absorption such as aluminum monostearate and gelatin.

In some cases, in order to prolong the effect of one or more compounds provided herein, it is desirable to slow the absorption of the compound from subcutaneous or intramuscular injection. For example, delayed absorption of a parenterally administered compound can be accomplished by dissolving or suspending the compound in an oil vehicle.

Compositions prepared as described herein can be administered in various forms, depending on the disorder to be treated and the age, condition, and body weight of the patient, as is well known in the art. For example, where the compositions are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These formulations can be prepared by conventional means in conjunction with the methods described herein, and, if desired, the active ingredient may be mixed with any conventional additive or excipient, such as a binder, a disintegrating agent, a lubricant, a corrigent, a solubilizing agent, a suspension aid, an emulsifying agent, or a coating agent.

Formulations suitable for oral administration may be in the form of capsules (e.g., gelatin capsules), cachets, pills, tablets, lozenges (using a flavored basis, usually sucrose and acacia or tragacanth), powders, troches, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound provided herein as an active ingredient. A composition may also be administered as a bolus, electuary, or paste. Oral compositions generally include an inert diluent or an edible carrier.

Pharmaceutical compositions suitable for parenteral administration can include one or more compounds provided herein in combination with one or more pharmaceutically acceptable sterile aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some embodiments, the pharmaceutical composition described herein comprise a surfactant. Suitable surfactants include pluronics, polysorbates, and the like. In some cases, the pharmaceutical composition described herein comprises a polysorbate, such as polysorbate 80. The surfactant can be present in an amount of about 0.01% (w/w) to about 20% (w/w), such as about 1%, 2%, 3%, 4%, 5%, 6%, 7%, 8%, 9%, 10%, or 15% (w/w). For example, the surfactant can be present in an amount of about 10%.

The composition should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the methods of preparation are freeze-drying (lyophilization), which yields a powder of the active ingredient plus any additional desired ingredient from a previously sterile-filtered solution thereof.

Actual dosage levels of the active ingredients in the pharmaceutical compositions provided herein may be varied so as to obtain “therapeutically effective amount,” which is an amount of the active ingredient effective to achieve the desired therapeutic response for a particular patient, composition, and mode of administration, without being toxic to the patient.

The concentration of a compound provided herein in a pharmaceutically acceptable composition will vary depending on several factors, including the dosage of the compound to be administered, the pharmacokinetic characteristics of the compound(s) employed, and the route of administration. In some embodiments, the compositions provided herein can be provided in an aqueous solution containing about 0.1-10% w/v of a compound disclosed herein, among other substances, for parenteral administration. Typical dose ranges can include from about 0.01 to about 50 mg/kg of body weight per day, given in 1-4 divided doses. Each divided dose may contain the same or different compounds. The dosage will be a therapeutically effective amount depending on several factors including the overall health of a patient, and the formulation and route of administration of the selected compound(s).

In some embodiments, the LMP2-selective inhibitor is administered in an amount of 1 to 40 mg/kg per day, e.g., 1 to 30, 5 to 20, 10 to 40, 15 to 35, 5 to 15, 10 to 25, 20 to 35, 25 to 40, or 1 to 10, mg/kg per day. In various embodiments, the LMP7-selective inhibitor is administered in an amount of 1 to 40 mg/kg per day, e.g., 1 to 30, 5 to 20, 10 to 40, 15 to 35, 5 to 15, 10 to 25, 20 to 35, 25 to 40, or 1 to 10, mg/kg per day etc.

The pharmaceutical composition may be administered at once, or may be divided into a number of smaller doses to be administered at intervals of time. It is also noted that the dose of the compound can be varied over time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the patient and adjusting the dosage and/or timing.

The pharmaceutical compositions can be included in a container, pack, or dispenser together with instructions for administration.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Other Embodiments

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

### EXAMPLES

The following examples are provided for illustration and are not intended to limit the scope of the invention.

### Methods

Mice. BALB/c mice (H-2^{d}) were purchased from Taconic Labs. All experiments were done under protocols approved by an institutional animal care and use committee

Arthritis model. Anti-collagen antibody induced arthritis (CAIA) was induced in 7-8 week old female BALB/c mice (kept on breeder chow) by an i.v. administration of 1.75 mg of a cocktail of 5 antibodies against type II collagen (Chondrex, Redmond, Wash.) followed by intraperitoneal challenge with 25 μg LPS on day 3. Treatment was initiated after clinical signs of arthritis were observed (day 4). Paws were scored for disease severity on a 0 (no disease)—4 (maximal swelling) scoring system and summed for individual animal scores.

Statistical analysis. For ANOVA, Bonferroni post hoc analysis was used to compare treatment groups. All statistical analyses were performed using GraphPad Prism Software (version 7.01). Statistical significance was achieved when P was less than 0.05.

Proteasome inhibition in mice. C-1057, C-3017 and ONX 0914 were all formulated in an aqueous solution of 10% (w/v) sulfobutylether-β-cyclodextrin and 10 mM sodium citrate (pH 3.5) and administered to mice as a single i.v. bolus. Whole blood (sodium heparin anti-coagulant) and tissue samples (kidney, and spleen) were collected 1 hr after administration and processed as described15 for protein quantitation and proteasome activity determination using the active site ELISA.

### Dual Inhibition of LMP7 and LMP2 is Required for Blocking Disease Progression in CAIA

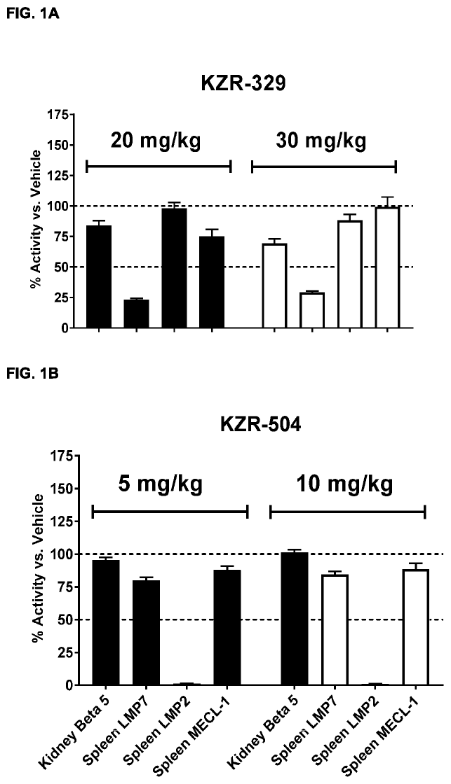

BALB/c mice received an i.v. administration of C-1057 at 20 mg/kg, C-3017 at 5 mg/kg or the combination of the two at 20 and 5 mg/kg. Whole blood, kidney and splenocytes samples were taken 1 hr after dosing and the activity of LMP7, LMP2, MECL1 (splenocytes) and β5 (kidney) were measured by active site ELISA. Data were normalized to the average activity of vehicle treated animals and are presented as the average relative activity+SEM (N=3). See FIG. 1A (C-1057 at 20 mg/kg and 30 mg/kg), FIG. 1B (C-3017 at 5 mg/kg and 10 mg/kg); and FIG. 1C (C-1057+C-3017 at 20/5 mg/kg and 20/10 mg/kg).

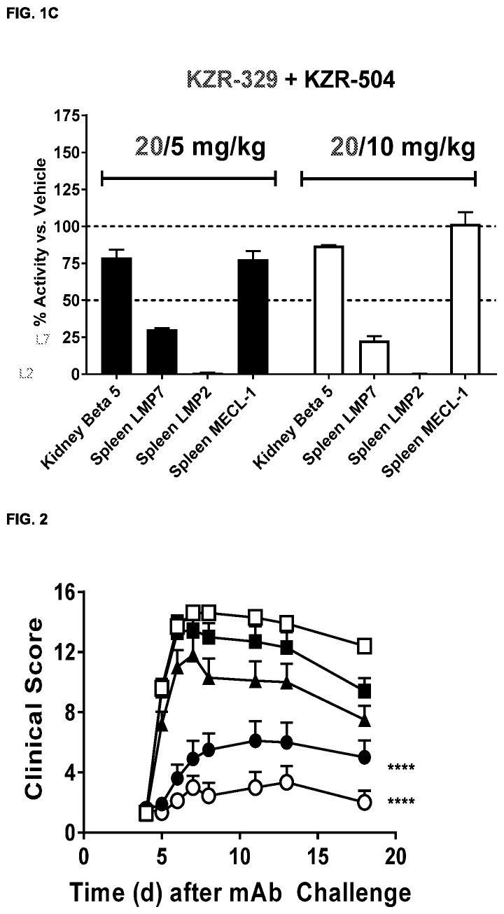

BALB/c mice received 1.75 mg of a cocktail of 5 antibodies against type II collagen on day 0 followed by 25 μg of LPS on day 3. On day 4, when disease symptoms were present in all mice, animals were randomized into 5 groups and were treated i.v. with either vehicle (□), C-1057 at 20 mg/kg (▴), C-3017 at 5 mg/kg (▮), ONX 0914 at 10 mg/kg (○) or the combination of C-1057 at 20 mg/kg and C-3017 at 5 mg/kg (●). Dosing was repeated on days 6, 8, 11, 13 and 15 and clinical scores (0-4/paw; N=7/group) were followed until day 18. Data, presented as the mean clinical score +SEM, are from one experiment of two performed with similar results. **=P<0.01, ***=P<0.001 or ****=P<0.0001 by two-way ANOVA followed by Bonferroni post-hoc comparison at the end of study. See FIG. 2.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step not specifically disclosed.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference.

We claim:

1. A pharmaceutical composition comprising: (a) a LMP2-selective inhibitor, (b) a LMP7-selective inhibitor; and (c) a pharmaceutically acceptable carrier.

2. A method of treating an immune-related disorder in a patient comprising administering to the patient in combination: (a) a therapeutically effective amount of an LMP2-selective inhibitor, and (b) a therapeutically effective amount of an LMP7-selective inhibitor.

3. The composition of claim 1 or the method of claim 2, wherein the LMP2-selective inhibitor has a structure of Formula (II), or a pharmaceutically acceptable salt thereof: 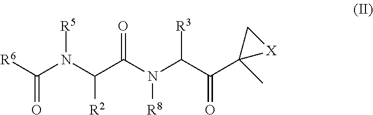
wherein:
X is selected from O, S, NH, and N—C₁₋₆alkyl;
R² and R³ are each independently selected from aryl, C₁₋₆aralkyl, heteroaryl, and _{c1-6}heteroaralkyl;
R⁵ is selected from hydrogen, OH, C₁₋₆aralkyl, and C₁₋₆alkyl; 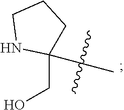 [SMILES: *C1(CO)CCCN1]
R⁶ is heteroaryl, piperidinyl, piperazinyl, morpholinyl, a lactone, a lactam, or and
R⁸ is selected from hydrogen, C₁₋₆alkyl, and C₁₋₆aralkyl.

4. The composition of claim 1 or the method of claim 2, wherein the LMP2-selective inhibitor has a structure selected from group consisting of: 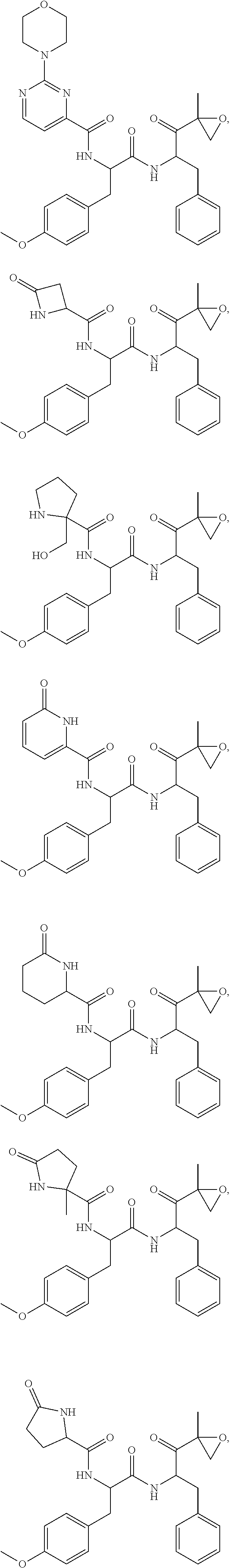
and a combination thereof, or a pharmaceutically acceptable salt thereof.

5. The composition or method of claim 4, wherein the LMP2-selective inhibitor is selected from the group consisting of: 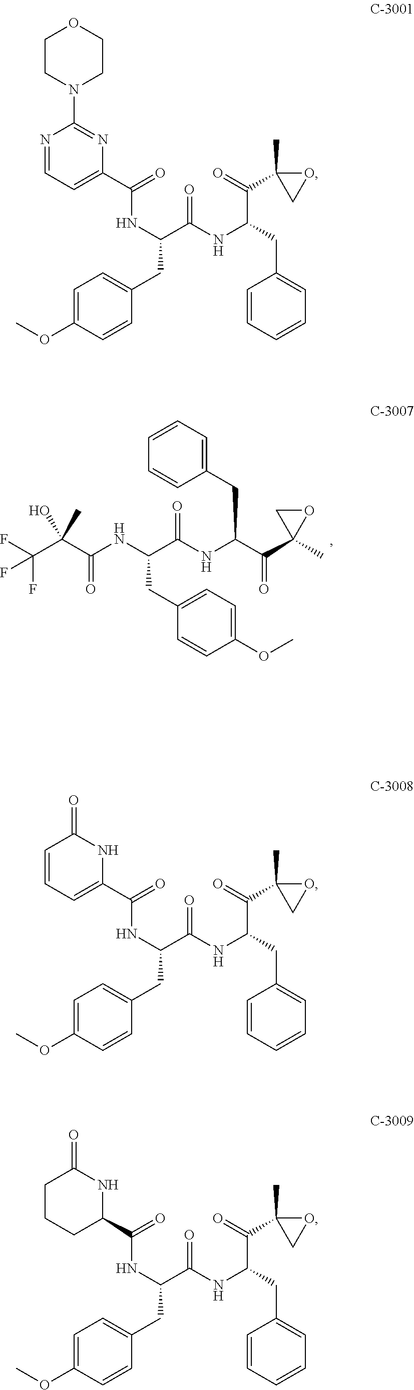 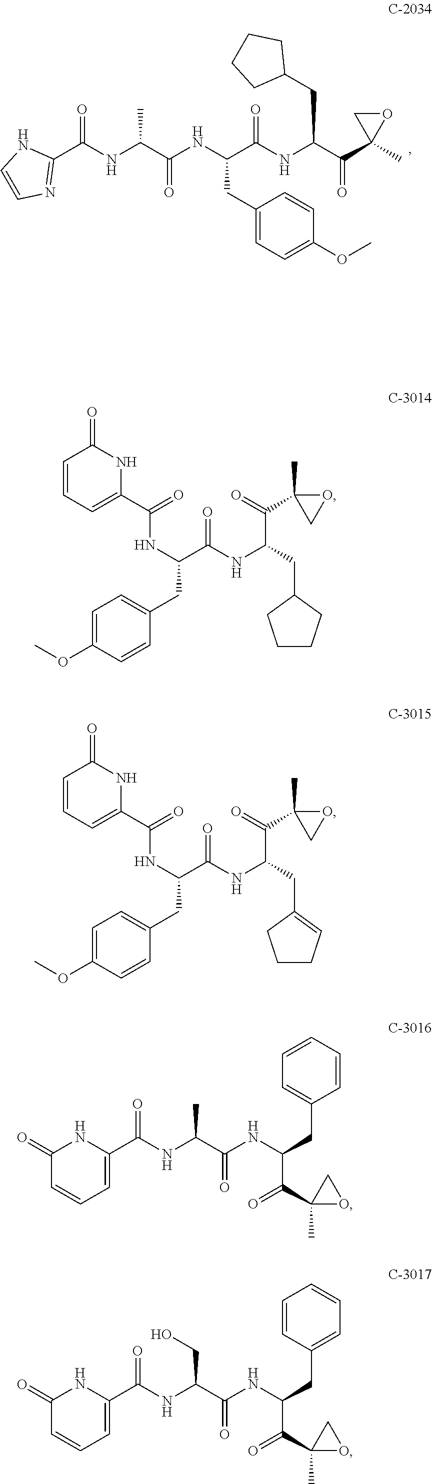 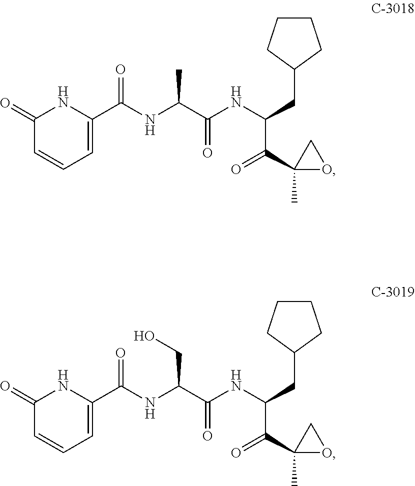 [SMILES: C[C@H](NC(=O)c1cccc(=O)[nH]1)C(=O)N[C@@H](CC1CCCC1)C(=O)[C@@]1(C)CO1 ; C[C@]1(C(=O)[C@H](CC2CCCC2)NC(=O)[C@H](CO)NC(=O)c2cccc(=O)[nH]2)CO1]
and a combination thereof, or a pharmaceutically acceptable salt thereof.

6. The composition or method of claim 5, wherein the LMP2-selective inhibitor is 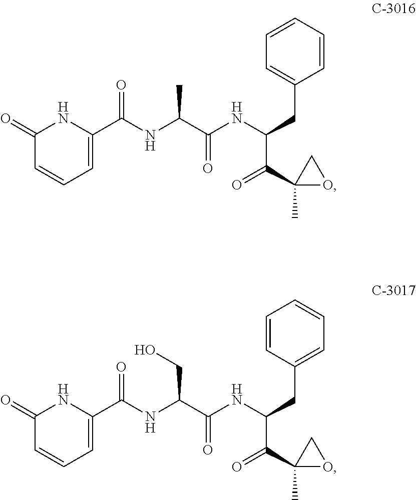 [SMILES: C[C@H](NC(=O)c1cccc(=O)[nH]1)C(=O)N[C@@H](Cc1ccccc1)C(=O)[C@@]1(C)CO1 ; C[C@]1(C(=O)[C@H](Cc2ccccc2)NC(=O)[C@H](CO)NC(=O)c2cccc(=O)[nH]2)CO1]
or a combination thereof, or a pharmaceutically acceptable salt thereof.

7. The composition or method of any one of claims 1-6, wherein the LMP7-selective inhibitor has a structure of Formula (X), or a pharmaceutically acceptable salt thereof: 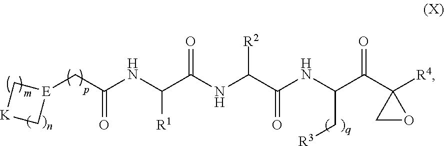
wherein:
m and n each independently are 0, 1 or 2, and m+n=2, 3, or 4;
p is 0 or 1;
q is 0, 1, or 2;
K is selected from the group consisting of CR⁵R⁶, NR⁷, N(C═O)OR⁷, —NH—(C═O)—,
O, S, SO, and SO₂;
E is N or CR⁷;
R¹ is selected from the group consisting of H, C₁₋₆alkyl, C₂₋₆alkenyl, C₂₋₆alkynyl, C₃₋₆cycloalkyl, and 3-6 membered heterocycloalkyl, wherein R¹ is optionally substituted with one or more substituents selected from the group consisting of halo, OR⁷, SR⁷, N(R⁷)₂, CN, and (C═O)N(R⁷)₂;
R² is C₁₋₂alkylene-G or (C═O)-G; wherein G is selected from the group consisting of aryl, heteroaryl, and pyridinone, with the proviso that when R² is CH₂phenyl, the phenyl is substituted with one or more substituents selected from the group consisting of OR⁷, halo, C₁₋₃alkyl, OCF₃, SO₂R⁷, (C═O)N(R⁷)₂, CN, and SO₂N(R⁷)₂;
R³ is non-aromatic and selected from the group consisting of C₃₋₇cycloalkyl, C₃₋₇cycloalkenyl, a 3-7 membered heterocycloalkyl, and a 3-7 membered heterocycloalkenyl, wherein R³ is optionally substituted with one or more substituents selected from the group consisting of halo, ═O, OR⁷, SR⁷, N(R⁷)₂, O(C═O)N(R⁷)₂, and C₁₋₆alkyl;
R⁴ is H or C₁₋₃alkyl;
R⁵ and R⁶ are each independently selected from the group consisting of H, OH, halo, C₁₋₃alkyl, and CF₃, or R⁵ and R⁶ together with the carbon to which they are attached form C═O or 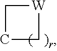
wherein W is O or NR⁷, and r is 1, 2 or 3; and
each R⁷ is independently H or C₁₋₆alkyl.

8. The composition or method of claim 7, wherein the LMP7-selective inhibitor has a structure selected from group consisting of: 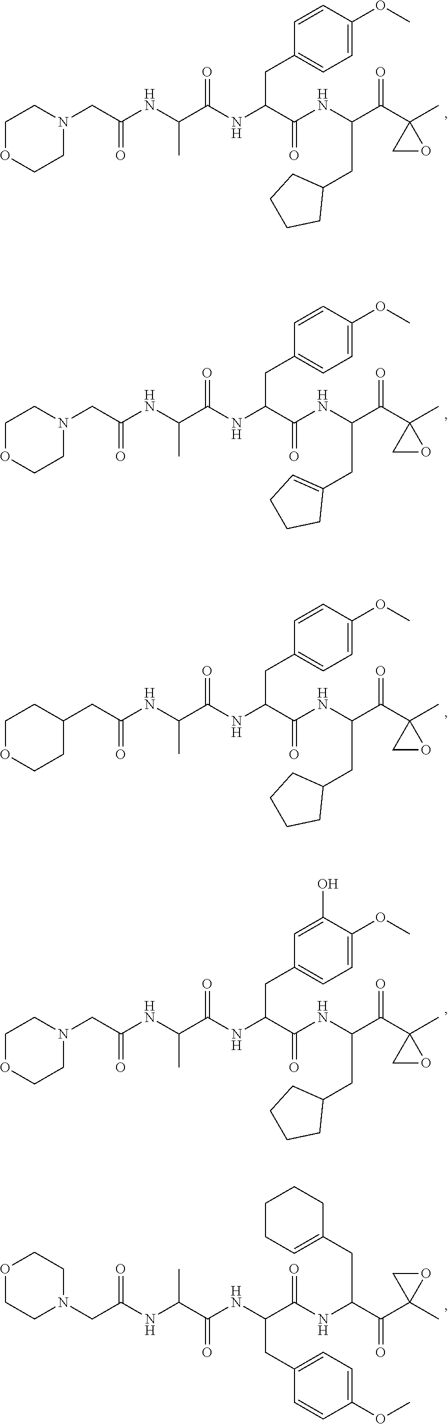 [SMILES: COc1ccc(CC(NC(=O)C(C)NC(=O)CN2CCOCC2)C(=O)NC(CC2CCCC2)C(=O)C2(C)CO2)cc1 ; COc1ccc(CC(NC(=O)C(C)NC(=O)CN2CCOCC2)C(=O)NC(CC2=CCCC2)C(=O)C2(C)CO2)cc1 ; COc1ccc(CC(NC(=O)C(C)NC(=O)CC2CCOCC2)C(=O)NC(CC2CCCC2)C(=O)C2(C)CO2)cc1 ; COc1ccc(CC(NC(=O)C(C)NC(=O)CN2CCOCC2)C(=O)NC(CC2CCCC2)C(=O)C2(C)CO2)cc1O ; COc1ccc(CC(NC(=O)C(C)NC(=O)CN2CCOCC2)C(=O)NC(CC2=CCCCC2)C(=O)C2(C)CO2)cc1] 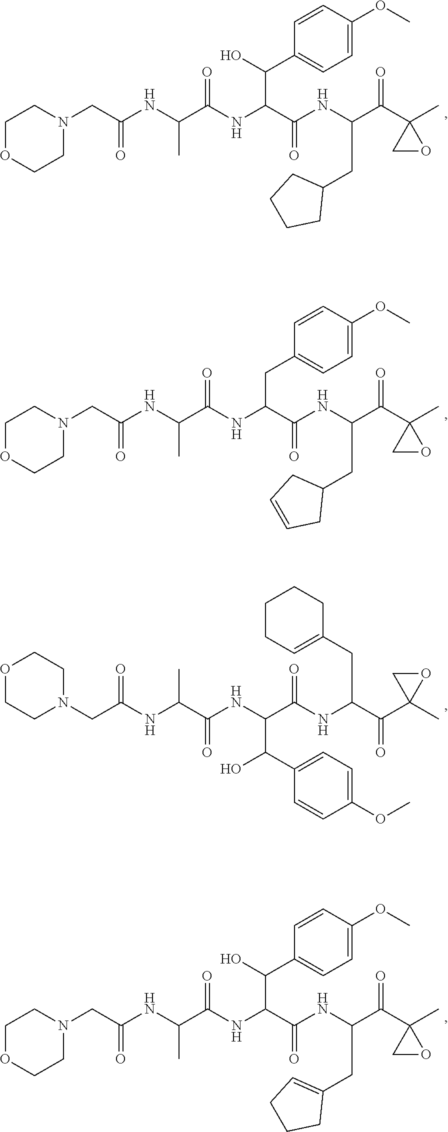 [SMILES: COc1ccc(C(O)C(NC(=O)C(C)NC(=O)CN2CCOCC2)C(=O)NC(CC2CCCC2)C(=O)C2(C)CO2)cc1 ; COc1ccc(CC(NC(=O)C(C)NC(=O)CN2CCOCC2)C(=O)NC(CC2CC=CC2)C(=O)C2(C)CO2)cc1 ; COc1ccc(C(O)C(NC(=O)C(C)NC(=O)CN2CCOCC2)C(=O)NC(CC2=CCCCC2)C(=O)C2(C)CO2)cc1 ; COc1ccc(C(O)C(NC(=O)C(C)NC(=O)CN2CCOCC2)C(=O)NC(CC2=CCCC2)C(=O)C2(C)CO2)cc1] 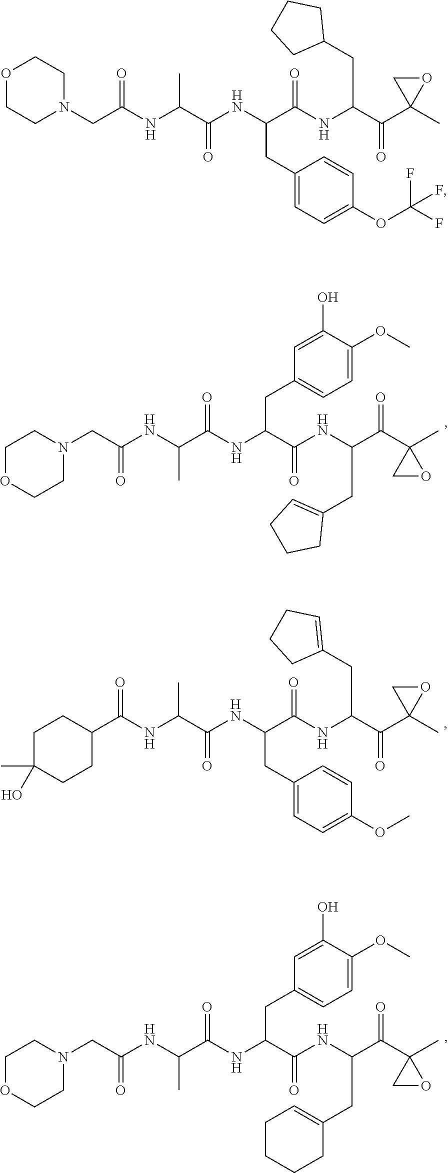 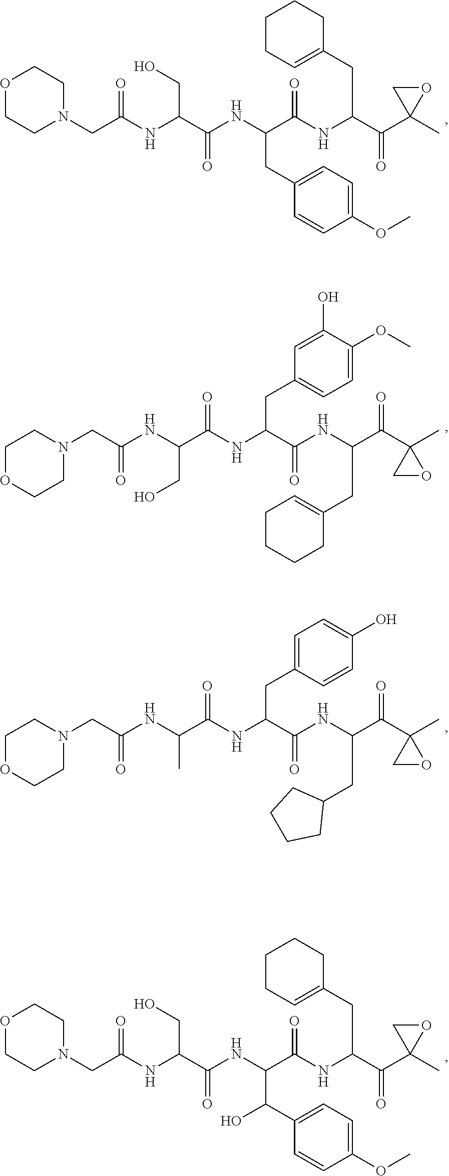 [SMILES: COc1ccc(CC(NC(=O)C(CO)NC(=O)CN2CCOCC2)C(=O)NC(CC2=CCCCC2)C(=O)C2(C)CO2)cc1 ; COc1ccc(CC(NC(=O)C(CO)NC(=O)CN2CCOCC2)C(=O)NC(CC2=CCCCC2)C(=O)C2(C)CO2)cc1O ; CC(NC(=O)CN1CCOCC1)C(=O)NC(Cc1ccc(O)cc1)C(=O)NC(CC1CCCC1)C(=O)C1(C)CO1 ; COc1ccc(C(O)C(NC(=O)C(CO)NC(=O)CN2CCOCC2)C(=O)NC(CC2=CCCCC2)C(=O)C2(C)CO2)cc1] 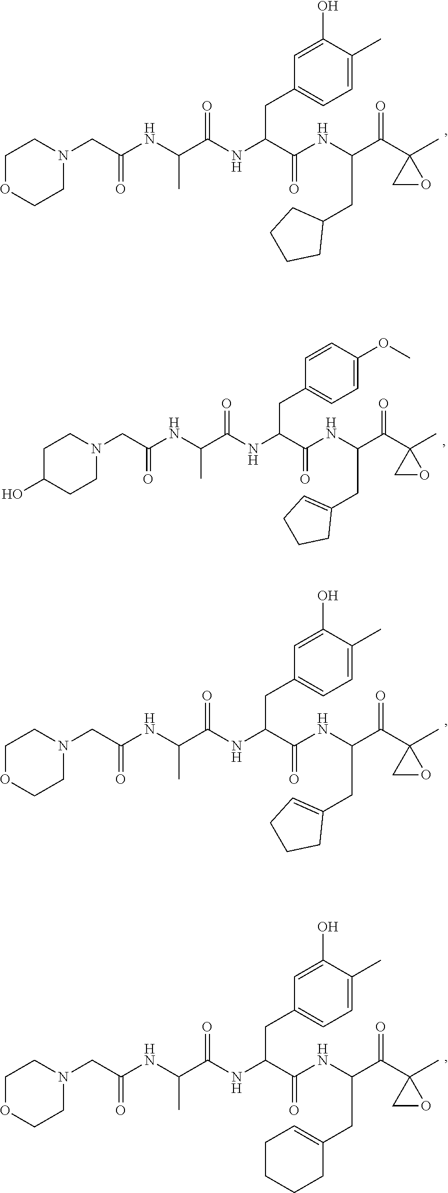 [SMILES: Cc1ccc(CC(NC(=O)C(C)NC(=O)CN2CCOCC2)C(=O)NC(CC2CCCC2)C(=O)C2(C)CO2)cc1O ; COc1ccc(CC(NC(=O)C(C)NC(=O)CN2CCC(O)CC2)C(=O)NC(CC2=CCCC2)C(=O)C2(C)CO2)cc1 ; Cc1ccc(CC(NC(=O)C(C)NC(=O)CN2CCOCC2)C(=O)NC(CC2=CCCC2)C(=O)C2(C)CO2)cc1O ; Cc1ccc(CC(NC(=O)C(C)NC(=O)CN2CCOCC2)C(=O)NC(CC2=CCCCC2)C(=O)C2(C)CO2)cc1O] 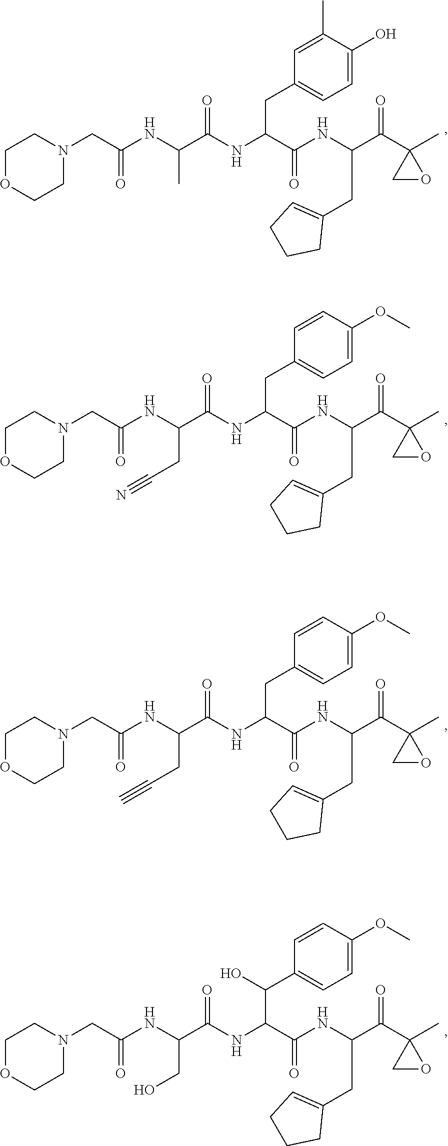 [SMILES: Cc1cc(CC(NC(=O)C(C)NC(=O)CN2CCOCC2)C(=O)NC(CC2=CCCC2)C(=O)C2(C)CO2)ccc1O ; COc1ccc(CC(NC(=O)C(CC#N)NC(=O)CN2CCOCC2)C(=O)NC(CC2=CCCC2)C(=O)C2(C)CO2)cc1 ; C#CCC(NC(=O)CN1CCOCC1)C(=O)NC(Cc1ccc(OC)cc1)C(=O)NC(CC1=CCCC1)C(=O)C1(C)CO1 ; COc1ccc(C(O)C(NC(=O)C(CO)NC(=O)CN2CCOCC2)C(=O)NC(CC2=CCCC2)C(=O)C2(C)CO2)cc1] 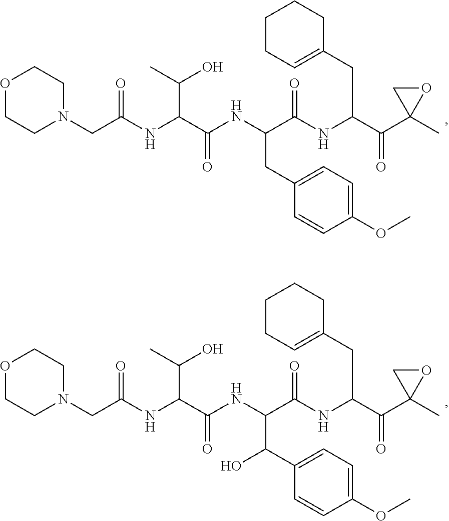 [SMILES: COc1ccc(CC(NC(=O)C(NC(=O)CN2CCOCC2)C(C)O)C(=O)NC(CC2=CCCCC2)C(=O)C2(C)CO2)cc1 ; COc1ccc(C(O)C(NC(=O)C(NC(=O)CN2CCOCC2)C(C)O)C(=O)NC(CC2=CCCCC2)C(=O)C2(C)CO2)cc1]
and a combination thereof, or a pharmaceutically acceptable salt thereof.

9. The composition or method of claim 8, wherein the LMP7-selective inhibitor is selected from the group consisting of: 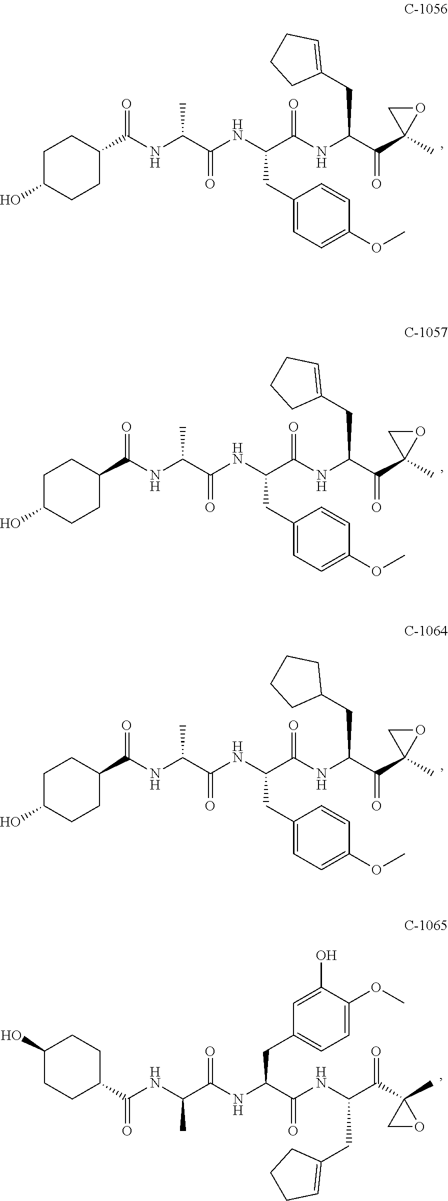 [SMILES: COc1ccc(C[C@H](NC(=O)[C@@H](C)NC(=O)[C@H]2CC[C@@H](O)CC2)C(=O)N[C@@H](CC2=CCCC2)C(=O)[C@@]2(C)CO2)cc1 ; COc1ccc(C[C@H](NC(=O)[C@@H](C)NC(=O)[C@H]2CC[C@H](O)CC2)C(=O)N[C@@H](CC2=CCCC2)C(=O)[C@@]2(C)CO2)cc1 ; COc1ccc(C[C@H](NC(=O)[C@@H](C)NC(=O)[C@H]2CC[C@H](O)CC2)C(=O)N[C@@H](CC2CCCC2)C(=O)[C@@]2(C)CO2)cc1 ; COc1ccc(C[C@H](NC(=O)[C@@H](C)NC(=O)[C@H]2CC[C@H](O)CC2)C(=O)N[C@@H](CC2=CCCC2)C(=O)[C@@]2(C)CO2)cc1O] 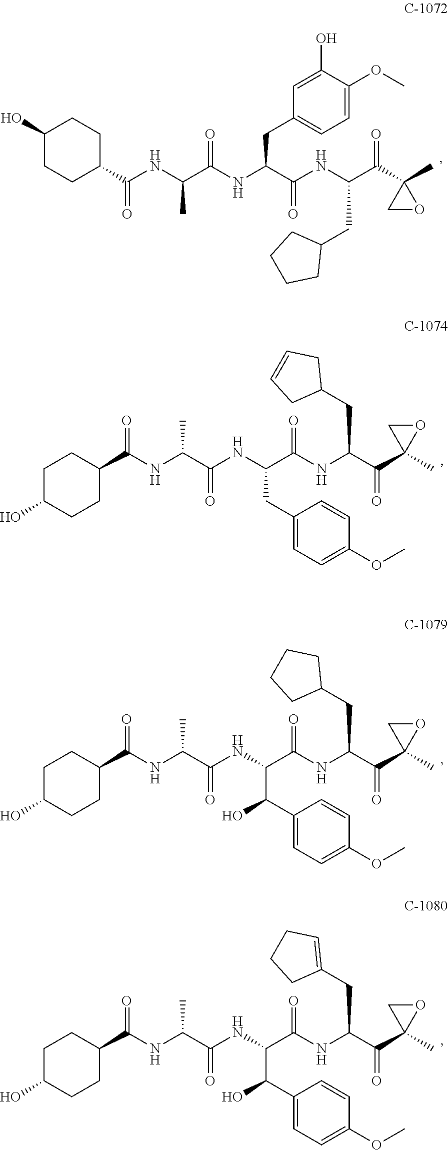 [SMILES: COc1ccc(C[C@H](NC(=O)[C@@H](C)NC(=O)[C@H]2CC[C@H](O)CC2)C(=O)N[C@@H](CC2CCCC2)C(=O)[C@@]2(C)CO2)cc1O ; COc1ccc(C[C@H](NC(=O)[C@@H](C)NC(=O)[C@H]2CC[C@H](O)CC2)C(=O)N[C@@H](CC2CC=CC2)C(=O)[C@@]2(C)CO2)cc1 ; COc1ccc([C@@H](O)[C@H](NC(=O)[C@@H](C)NC(=O)[C@H]2CC[C@H](O)CC2)C(=O)N[C@@H](CC2CCCC2)C(=O)[C@@]2(C)CO2)cc1 ; COc1ccc([C@@H](O)[C@H](NC(=O)[C@@H](C)NC(=O)[C@H]2CC[C@H](O)CC2)C(=O)N[C@@H](CC2=CCCC2)C(=O)[C@@]2(C)CO2)cc1] 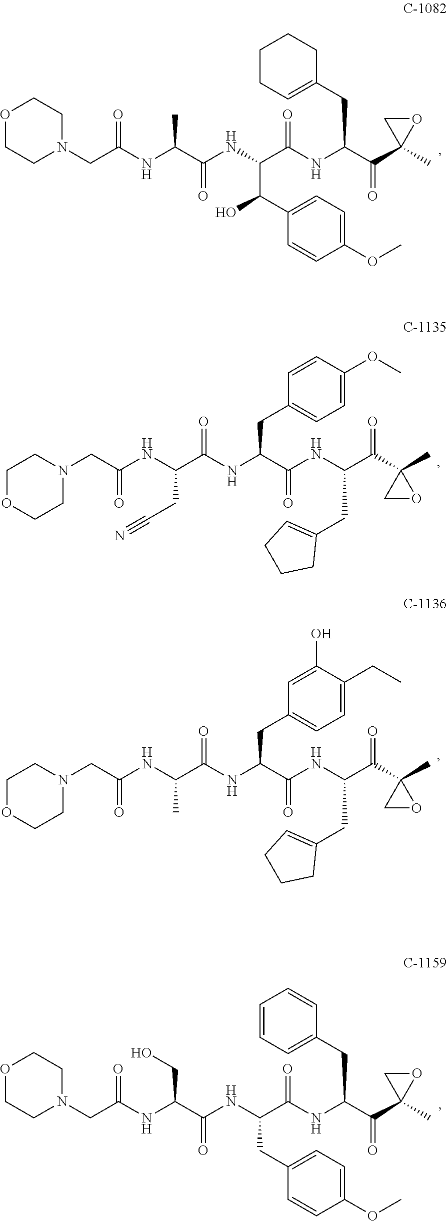 [SMILES: COc1ccc([C@@H](O)[C@H](NC(=O)[C@H](C)NC(=O)CN2CCOCC2)C(=O)N[C@@H](CC2=CCCCC2)C(=O)[C@@]2(C)CO2)cc1 ; COc1ccc(C[C@H](NC(=O)[C@H](CC#N)NC(=O)CN2CCOCC2)C(=O)N[C@@H](CC2=CCCC2)C(=O)[C@@]2(C)CO2)cc1 ; CCc1ccc(C[C@H](NC(=O)[C@H](C)NC(=O)CN2CCOCC2)C(=O)N[C@@H](CC2=CCCC2)C(=O)[C@@]2(C)CO2)cc1O ; COc1ccc(C[C@H](NC(=O)[C@H](CO)NC(=O)CN2CCOCC2)C(=O)N[C@@H](Cc2ccccc2)C(=O)[C@@]2(C)CO2)cc1] 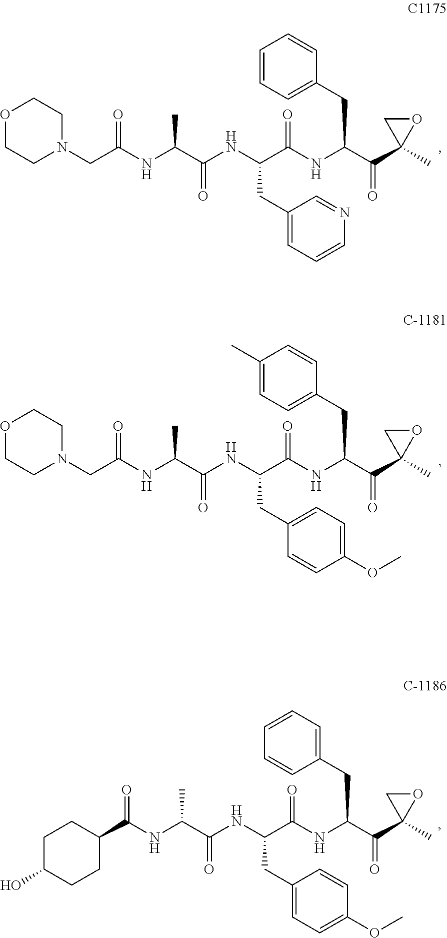 [SMILES: C[C@H](NC(=O)CN1CCOCC1)C(=O)N[C@@H](Cc1cccnc1)C(=O)N[C@@H](Cc1ccccc1)C(=O)[C@@]1(C)CO1 ; COc1ccc(C[C@H](NC(=O)[C@H](C)NC(=O)CN2CCOCC2)C(=O)N[C@@H](Cc2ccc(C)cc2)C(=O)[C@@]2(C)CO2)cc1 ; COc1ccc(C[C@H](NC(=O)[C@@H](C)NC(=O)[C@H]2CC[C@H](O)CC2)C(=O)N[C@@H](Cc2ccccc2)C(=O)[C@@]2(C)CO2)cc1]
and a combination thereof, or a pharmaceutically acceptable salt thereof.

10. The composition or method of claim 9, wherein the LMP7-selective inhibitor is selected from the group consisting of C-1056, C-1057, C-1064, C-1065, C-1072, C-1074, C-1079, C-1080, C-1186, and a combination thereof, or a pharmaceutically acceptable salt thereof.

11. The composition of claim 1 or the method of claim 2, wherein the LMP2selective inhibitor is C-3016, or a pharmaceutically acceptable salt thereof.

12. The composition of claim 1 or the method of claim 2, wherein the LMP7-selective inhibitor is C-3017, or a pharmaceutically acceptable salt thereof.

13. The composition or method of claim 11 or 12, wherein the LMP7-selective inhibitor is C-1056, or a pharmaceutically acceptable salt thereof.

14. The composition or method of claim 11 or 12, wherein the LMP7-selective inhibitor is C-1057, or a pharmaceutically acceptable salt thereof.

15. The composition or method of claim 11 or 12, wherein the LMP7-selective inhibitor is C-1064, or a pharmaceutically acceptable salt thereof.

16. The composition or method of claim 11 or 12, wherein the LMP7-selective inhibitor is C-1065, or a pharmaceutically acceptable salt thereof.

17. The composition or method of claim 11 or 12, wherein the LMP7-selective inhibitor is C-1072, or a pharmaceutically acceptable salt thereof.

18. The composition or method of claim 11 or 12, wherein the LMP7-selective inhibitor is C-1074, or a pharmaceutically acceptable salt thereof.

19. The composition or method of claim 11 or 12, wherein the LMP7-selective inhibitor is C-1079, or a pharmaceutically acceptable salt thereof.

20. The composition or method of claim 11 or 12, wherein the LMP7-selective inhibitor is C-1080, or a pharmaceutically acceptable salt thereof.

21. The composition or method of claim 11 or 12, wherein the LMP7-selective inhibitor is C-1186, or a pharmaceutically acceptable salt thereof.

22. The method of any one of claims 2-21, wherein the LMP2-selective inhibitor and the LMP7-selective inhibitor are each administered in a pharmaceutical composition.

23. The composition or method of any one of claims 1 and 3-22, wherein the pharmaceutically acceptable carrier comprises a cyclodextrin.

24. The composition or method of claim 23, wherein the cyclodextrin is beta-cyclodextrin, sulfobutylether-beta-cyclodextrin, or (2-hydroxypropyl)-beta-cyclodextrin.

25. The composition or method of any one of claims 22-24, wherein the pharmaceutical composition comprises a surfactant.

26. The composition or method of claim 25, wherein the surfactant comprises a polysorbate.

27. The composition or method of claim 26, wherein the surfactant comprises polysorbate 80.

28. The composition or method of any one of claims 25-27, wherein the surfactant is present in an amount of about 10% (w/w).

29. The method of any one of claims 2-28, wherein the LMP2-selective inhibitor and the LMP7-selective inhibitor are administered simultaneously.

30. The method of claim 29, wherein the LMP2-selective inhibitor and the LMP7-selective inhibitor are administered in the same pharmaceutical composition.

31. The method of any one of claims 2-28, wherein the LMP2-selective inhibitor and the LMP7-selective inhibitor are administered sequentially.

32. The method of any one of claims 2-31, wherein the LMP2-selective inhibitor and the LMP7-selective inhibitor are administered orally or parenterally.

33. The method of claim 32, wherein at least one of the LMP2-selective inhibitor and the LMP7-selective inhibitor is administered subcutaneously.

34. The method of claim 33, wherein each of the LMP2-selective inhibitor and the LMP7-selective inhibitor is administered subcutaneously.

35. The method of any one of claims 32-34, wherein the LMP2-selective inhibitor and the LMP7-selective inhibitor are administered via injection and at the same injection site.

36. The method of any one of claims 32-34, wherein the LMP2-selective inhibitor and the LMP7-selective inhibitor are administered via injection at different injection sites.

37. The method of any one of claims 2-36, wherein the immune-related disorder is selected from the group consisting of rheumatoid arthritis, lupus, multiple sclerosis, psoriasis, chronic obstructive pulmonary disease (“COPD”), granulomatosis and vasculitis, graft or transplant-related disease, and fibrotic disease.

38. The method of claim 37, wherein the disorder is rheumatoid arthritis, lupus, multiple sclerosis, psoriasis, COPD, or granulomatosis and vasculitis.

39. The method of claim 37 or 38, wherein the lupus is systemic lupus erythematosus (“SLE”), subacute cutaneous lupus, drug-induced lupus, neonatal lupus, or discoid lupus.

40. The method of claim 37, wherein the fibrotic disease is selected from the group consisting of cystic fibrosis, idiopathic pulmonary fibrosis (“IPF”), cirrhosis, biliary atresia, atrial fibrosis, endomyocardial fibrosis, arterial stiffness, arthrofibrosis, Crohn's Disease, Dupuytren's contracture, keloid, mediastinal fibrosis, myelofibrosis, Peyronie's disease, nephrogenic systemic fibrosis, progressive massive fibrosis, retroperitoneal fibrosis, and scleroderma.
